(19) United States
(12) Patent Application Publication
(10) Pub. No.: US 2014/0206572 A1
(43) Pub. Date: Jul. 24, 2014

(54) OVARIAN MARKERS OF FOLLICULAR MATURITY AND USES THEREOF

(71) Applicants: Marc-Andre Sirard, Quebec (CA); Anne-Laure Nivet, Quebec (CA); Audrey Bunel, Quebec (CA); Remi Labrecque, St-Bernard (CA)

(72) Inventors: Marc-Andre Sirard, Quebec (CA); Anne-Laure Nivet, Quebec (CA); Audrey Bunel, Quebec (CA); Remi Labrecque, St-Bernard (CA)

(73) Assignee: UNIVERSITE LAVAL, Quebec, QC (CA)

(21) Appl. No.: 14/234,671

(22) PCT Filed: Jul. 24, 2012
(86) PCT No.: PCT/CA2012/000697

§ 371 Date: Mar. 18, 2014

Related U.S. Application Data
(60) Provisional application No. 61/512,574, filed on Jul. 28, 2011.

(51) Int. Cl. G01N 33/68; G01N 33/569; G01N 33/76; C12Q 1/68

(52) U.S. Cl. CPC G01N 33/689; C12Q 1/6883; G01N 33/56966; G01N 33/76
USPC 506/9; 435/6.12; 435/7.92; 435/7.1; 435/6.11; 435/7.21

(57) ABSTRACT

The present invention relates to field of fertility. The invention identifies biological ovarian markers from follicular cells, from follicular fluid, from cumulus cells and from oocytes which are indicative of follicular maturity in mammals. Described are methods for improving ovarian stimulation, methods for assessing maturity of a mammalian ovarian follicle, methods for optimizing in vitro maturation (IVM) and methods for classifying an embryo, these methods being based on assessment of expression level of ovarian markers indicative of maturity. Also described are methods for screening compounds stimulatory of or inhibitory to mammalian follicular maturation, kits for evaluating follicular maturity.

### FIELD OF THE INVENTION

The present invention relates to field of fertility. More particularly, it relates to oocyte(s) and follicular cells markers of mammalian ovarian follicular maturity and their uses.

### BACKGROUND OF THE INVENTION

Oocyte's quality largely depends on the follicle from which it originates, as shown in a number of animal and human studies. During the in vitro fertilization (IVF) procedure upon ovarian stimulation and ovulation induction, a cohort of heterogeneous follicles is recruited to develop and ovulate, irrespective of their differentiate state. This creates an asynchrony in the maturation process and heterogeneity in the quality of the oocytes recovered for assisted reproduction. To determine the factors associated with the developmental competence of the oocytes and to understand how they influence the oocyte quality, follicles with different oocyte quality must be analyzed for these factors at the protein and gene levels.

Previous studies have tended to focus upon the appearance of the embryo (morphology) to predict the success of fertilization in vitro. Other means of investigate the embryo quality may interfere with embryo viability leading to an absence of objective criteria to distinguish between several embryos, which to transfer to the mother. In recent years, scientific evidences obtained both from animal models and humans are supporting the hypothesis that the oocyte quality and therefore its ability to implant post transfer depends on the follicular conditions prevailing in the ovary before the oocytes are removed. This leads to a method of predicting the outcome of IVF which involved firstly determining the level of target compounds in a biological sample taken from a female patient and then predicting, from the level of the compounds determined, the probability of establishing pregnancy in the subject by IVF. The level measured for a pool of cells from different follicles (from the same individual) was not always a true reflection of the level in individual follicles, suggesting that one or more follicles possess compounds affecting the probability of establishing a pregnancy.

In humans, IVF is normally done with in vivo matured oocytes whereas in animals such as bovine, IVF is normally done with in vitro matured (IVM) oocytes. IVM has been developed in cows in the mid-eighties with the use of slaughterhouse ovaries from unstimulated cows. Despite hundreds of technical papers on the improvement of culture conditions since then, the average developmental rate to blastocyst has not changed significantly. This situation has changed with the introduction of the follicle-stimulating hormone (FSH) coasting—i.e., FSH withdrawal—introduced a few years ago (Blondin et al., 2002 Biol Reprod 66: 38-43). It is known that maximal oocyte competence acquisition occurs in large animals, including cows, between the FSH surge and the pre-ovulation luteinizing hormone (LH) surge (Sirard et al., 2006, Theriogenology 65: 126-136). Assuming that a period between FSH and LH surge is favorable for oocyte competence, the optimal competence acquisition window calls for a better characterization of FSH coasting conditions by defining the conditions prior to and following this change, in other words the borders of the window of competence. There is thus a need for biological markers associated with follicular maturity status, a need for methods for assessing maturity of a mammalian ovarian follicles and a need for methods for optimizing assisted reproduction techniques, particularly in humans.

International PCT Patent publications WO 2007/130673 and WO 2008/066655 describe a series of oocyte, follicular fluid, and/or cumulus cells markers for evaluating the competence of a mammalian oocyte. International PCT Patent publication no. WO 2008/031226 describes using granulosa markers for determining oocyte competence. International PCT Patent publication no WO 2011/057411 describes ovarian markers for determining oocyte competence. Scientific publications by the inventors also describe marker genes as pregnancy predictors (Hamel et. al., (2010) Mol. Hum. Reprod., Vol. 16, No. 8, pp. 548-556; Assidi M, Montag M, Van Der Ven K, Sirard M A. Biomarkers of human oocyte developmental competence expressed in cumulus cells before ICSI: a preliminary study. J Assist Reprod Genet. 2010 Oct. 16). However, none of these documents provides biological markers and characterization methods for determining the maturity of ovarian follicles. Furthermore none of these documents provides methods for determining the best follicle(s), in term of embryo development, between two or more medium size follicles.

### SUMMARY OF THE INVENTION

The present invention contemplates the use of oocytes markers and follicular cells markers (including follicular fluid, cumulus cells and granulosa cells) for evaluating the maturity of mammalian ovarian follicle for numerous assisted reproduction techniques, for implantation and pregnancy induction or both.

As described hereinafter, the inventors have identified a series of biological ovarian markers associated with ovarian follicular maturity status. The identity of these biological ovarian markers is provided in Tables I, II and III.

One aspect of the invention concerns a method for optimizing assisted reproduction (AR) in mammals, preferably a human subject. In one embodiment the method comprises:
obtaining an oocyte and/or follicular cell(s) from an ovarian follicle subsequent to a first controlled ovarian stimulation (COS);
assessing maturity of said ovarian follicle by determining expression level of at least one ovarian marker indicative of said maturity;
optimizing a second COS based on the assessed maturity of the ovarian follicle obtained following the first COS.

Another aspect of the invention concerns a method for improving ovarian stimulation in a human subject, the method comprising:
obtaining an oocyte and/or follicular cell(s) from an ovarian follicle subsequent to a first controlled ovarian stimulation (COS);
assessing maturity of said ovarian follicle by determining expression level of at least one ovarian marker indicative of said maturity;
optimizing a second COS based on the assessed maturity of the ovarian follicle obtained following the first COS;
   wherein the second COS provides for an improved ovarian stimulation when compared to the first COS.

In one particular embodiment, the optimization of the second COS (as well as subsequent COS) comprises increasing or reducing dosage of hormone(s) (e.g. LH, FSH) administered to the human subject during COS. In one particular embodiment, the optimization comprises aspirating follicles for AR after a period of time following hormone stimulation shorter or longer than the period of time following hormone stimulation of the first COS, thereby aiming aspiration of follicles at an “ideal” maturity status.

Another aspect of the invention concerns a method of assessing maturity of a mammalian ovarian follicle comprising assessing expression of at least one ovarian marker (e.g. a polynucleotide or a polypeptide) from that ovarian follicle. The follicle may be from a human oocyte. The polynucleotide may be a DNA or a RNA sequence. The ovarian marker is selected from the genes listed in Tables I, II and III, and combinations thereof. Particular embodiments comprises assessing specifically cumulus cells marker(s). Particular embodiments comprises assessing specifically granulosa cells marker(s). Particular embodiments comprises assessing specifically oocyte marker(s). Particular embodiments comprises assessing expression of at least 2, 3, 5 or more markers and/or assessing expression of marker(s) from at least two different sources of biological material. In particular embodiments the ovarian marker is selected from PDE8B, THBD, TLR2, CHODL, TGFBR2 and combinations thereof.

In accordance with another embodiment the ovarian marker is a follicular cell marker which is expressed in follicular cells comprised in the ovarian follicle. In some embodiments, the preferred follicular cell markers include markers expressed in granulosa cells. Preferred granulosa cell markers include the genes listed in Table I, and combinations thereof. In other embodiments, the preferred follicular cell markers include markers expressed in cumulus cells originating from the follicle. Preferred cumulus cell markers include the genes listed in Table II and combinations thereof.

In accordance with one embodiment the ovarian marker is a oocyte marker which is expressed in an oocyte comprised in the follicle. Preferred oocyte markers include the genes listed in Table III and combinations thereof.

In preferred embodiments, the methods of the invention comprises comparing the expression level of the at least one marker with a control expression level. Assessment of the expression of the marker may comprises measuring polynucleotide and/or polypeptide expression levels for the marker. Examples of polynucleotides and polypeptide to be measured includes sequence as set forth on the NCBI's web site (i.e. “Gene”, the reference sequence collection of the NCBI's web site available on the internet at http://www.ncbi.nlm.nih.gov/gene/) for the GeneID provided in Tables I, II and

Another aspect of the invention concerns a method for assessing maturity of a mammalian ovarian follicle, the method comprising assessing expression of at least one follicular cell marker which is expressed in granulosa cells of the ovarian follicle, the expression level of the granulosa cell marker(s) being indicative of follicular maturity. The granulosa cell marker is selected from the genes listed in Table I and combinations thereof. Assessment of the expression of the at least one granulosa cell marker may comprises measuring polynucleotide (e.g. DNA and/or RNA levels) and/or polypeptide expression levels for said cumulus cell marker(s). In particular embodiments, the granulosa cell marker is selected from PDE8B, THBD, TLR2, CHODL, TGFBR2 and combinations thereof.

In one particular embodiment, the method of assessing maturity of a mammalian ovarian follicle comprises:
(a) assessing in granulosa cell(s) originating from the ovarian follicle an expression level of at least one polynucleotide, wherein the at least one polynucleotide comprises a nucleotide sequence as set forth in NCBI for the GeneID numbers provided in Table I; and
(b) comparing the expression level of the at least one polynucleotide with a control expression level;
wherein a differential between expression level of the at least one polynucleotide and the control expression level is indicative of follicular maturity status.

In another particular embodiment, the method of assessing maturity comprises:
(a) assessing in granulosa cells originating from the ovarian follicle an expression level of at least one polypeptide, wherein said polypeptide comprises an amino acid sequence as set forth in NCBI for the GeneID numbers provided in Table I; and
(b) comparing the expression level of the at least one polypeptide with a control expression level;
   wherein a differential between expression level of the at least one polypeptide and the control expression level is predicative of follicular maturity status.

Another aspect of the invention concerns a method for assessing maturity of a mammalian ovarian follicle, the method comprising assessing expression of at least one follicular cell marker which is expressed in cumulus cells originating from the ovarian follicle, the expression level of the follicular cell marker(s) being indicative of follicular maturity. The cumulus cell marker is selected from the genes listed in Table II and combinations thereof. Assessment of the expression of the at least one cumulus cell marker may comprises measuring polynucleotide (e.g. DNA and/or RNA levels) and/or polypeptide expression levels for said cumulus cell marker(s).

According to a particular embodiment, the method of assessing maturity of a mammalian ovarian follicle comprises:
(a) assessing in cumulus cell(s) originating from the ovarian follicle an expression level of at least one polynucleotide, wherein the polynucleotide comprises a nucleotide sequence as set forth in NCBI for the GeneID numbers provided in Table II; and
(b) comparing the expression level of the at least one nucleotide with a control expression level;
   wherein a differential between expression level of the at least one nucleotide and the control expression level is predicative of follicular maturity status.

According to another particular embodiment, the method assessing maturity comprises:
(a) assessing in cumulus cell(s) originating from the ovarian follicle an expression level of at least one polypeptide, wherein the polypeptide comprises an amino acid sequence as set forth in NCBI for the GeneID numbers provided in Table II; and
(b) comparing the expression level of the at least one polypeptide with a control expression level;
   wherein a differential between expression level of the at least one polypeptide and the control expression level is predicative of follicular maturity status.

Another aspect of the invention concerns a method for assessing maturity of a mammalian ovarian follicle, the method comprising assessing expression of at least one oocyte marker which is expressed in an oocyte comprised in the follicle, the expression level of the oocyte marker being indicative of follicular maturity. The oocyte marker is selected from the genes listed in Table III and combinations thereof. Assessment of the expression of the at least one oocyte marker may comprises measuring polynucleotide (e.g. DNA and/or RNA levels) and/or polypeptide expression levels for said cumulus cell marker(s).

According to a particular embodiment, the method of assessing maturity of a mammalian ovarian follicle comprises:
(a) assessing in an oocyte comprised in the follicle an expression level of at least one polynucleotide, wherein the polynucleotide comprises a nucleotide sequence as set forth in NCBI for the GeneID numbers provided in Table III; and
(b) comparing the expression level of the at least one nucleotide with a control expression level;
   wherein a differential between expression level of the at least one nucleotide and the control expression level is predicative of follicular maturity status.

According to a particular embodiment, the method of assessing maturity of a mammalian ovarian follicle comprises:
(a) assessing in an oocyte comprised in the follicle an expression level of at least one polypeptide, wherein the polypeptide wherein the polypeptide comprises an amino acid sequence as set forth in NCBI for the GeneID numbers provided in Table III; and
(b) comparing the expression level of the at least one polypeptide with a control expression level;
   wherein a differential between expression level of the at least one polypeptide and the control expression level is predicative of follicular maturity status.

The methods of the invention may further comprises comparing the expression level with expression level of control follicular cell(s) (including follicular fluid) and/or oocyte(s) and showing a significant change by using ratios or absolute amount to reflect follicular maturity. Control expression levels and ratios may also be calculated using housekeeping genes.

Another aspect of the invention concerns a method for classifying an embryo to be transferred after in vitro fertilization, comprising
obtaining granulosa cells from an ovarian follicle comprising an oocyte;
assessing maturity status of said ovarian follicle by determining expression level of at least one ovarian marker indicative of follicle maturity;
obtaining an embryo from said oocyte; and
classifying said embryo as a transferable or not-transferrable embryo based on the assessed follicular maturity.

In one embodiment, the method further comprises an additional step of transferring an embryo classified as a transferable embryo. Preferably, the transferable embryo originates from an oocyte obtained from a medium size follicle. Accordingly, the invention may also be useful for selecting “high” quality embryos, that is embryos that possess the desired ability to be transferred into a female recipient.

Another aspect of the invention concerns a method for optimizing assisted reproduction techniques in a human subject. In one embodiment the method is for optimising in vitro maturation (IVM)) and comprises:
obtaining cumulus cell(s) and/or granulosa cell(s) from an ovarian follicle comprising an oocyte;
assessing maturity status of the ovarian follicle by determining expression level of at least one ovarian marker indicative of the follicle's maturity;
selecting in vitro maturation (IVM) conditions favorable for maturation of said oocyte, these IVM conditions being adapted according to the maturity status of the follicle from which the oocyte originates;
   wherein said IVM conditions provides an optimized in vitro maturation.

Other aspects of the invention concerns methods for screening a compound stimulatory or inhibitory to mammalian follicular maturation and in vivo methods for assessing a compound activity to stimulate or inhibit follicular maturity in a subject.

Also provided is a kit for use in evaluating competence of mammalian oocytes. An array of nucleic acid probes immobilized on a solid support is also described.

An advantage of the invention is that it provides tools for assessing maturity of a mammalian ovarian follicle and for optimizing control ovarian stimulation (COS) protocols in order to obtain competent oocytes for assisted reproduction (AR) and maximize fertilization, embryo viability, embryo development and/or embryo implantation. The markers of the invention serve as indicators of successful ovarian hormonal stimulation regimen and they are a useful diagnostic tool to refine hormonal treatment of a patient or a population of patients. In addition, the markers of the invention may be helpful in optimizing in vitro maturation (IVM) media, both in terms of type and levels of components, and in optimizing IMV conditions and protocols.

Additional aspects, advantages and features of the present invention will become more fully understood from the detailed description given herein and from the accompanying figures, which are exemplary and should not be interpreted as limiting the scope of the invention.

### BRIEF DESCRIPTION OF THE FIGURES

FIG. 1 is a line graph showing blastocyst rate per cow and coasting period, according to the results of Example 1. Each symbol represents one cow. Blastocyst rate is expressed as a percentage. H=hours.

FIG. 2 is a box plot showing blastocyst rate dispersion per coasting period according to the results of Example 1. For each coasting period, from the bottom: minimal value, first quartile, median, third quartile, maximal value. Dotted line=mean.

FIG. 3 is a graph representing a mathematical model of the blastocyst probability according to the results of Example 1. Coasting was considered as a continual variable, and embryo as a binary variable, blastocyst at D8 or not, in a GEE model. The equation of blastocyst probability modelization was Log(p/1−p)=−1.2353+0.0757 coasting−0.0007 coasting*coasting. With this consideration, the coasting duration maximizing blastocyst rate (69%) was 54.07+/−7.71 hours.

FIG. 4 is a graph showing follicle size groups and coasting period, according to the results of Example 1. The proportion of each follicle size group is associated to a motif. Data with a common superscript do not differ significantly (p>0.050).

FIG. 5 is a line graph showing follicles, COCs, and theoretical blastocyst absolute data per coasting period, according to the results of Example 1.

FIGS. 6A, 6B and 6C are box plots showing follicle size group proportion dispersion per coasting period, according to the results of Example 1. For each coasting period, from the bottom: minimal value, first quartile, median, third quartile, maximal value. A: >10-mm follicles, B: 7-10-mm follicles, C: 5-6-mm follicles.

FIG. 7 are bar graphs illustrating quantitative PCR validation of selected granulosa-genes in bovine. Significant differences are indicated by different superscript letters. Coasting period is in hours.

FIG. 8A is a bar graph representing relative proportion of 4 follicle size groups and the 3 developmental competence groups, according to Example 6.

FIG. 8B is a line graph illustrating number of follicles in each developmental competence group in relation with follicle size groups, according to Example 6.

FIG. 9 is a panel illustrating microarray experimental design (human samples) according to Example 6. Each black circle represents a follicle, Plus (+) inside circle: transferable follicle, Minus (−) inside circle: follicle associated to oocyte with a development up to 10 cells. Black arrow: a dye swap.

FIG. 10 are bar graphs illustrating quantitative PCR validation of selected markers in granulosa cells according to Example 6. m+: medium size follicles associated to transferable embryos, m−: medium size follicles not associated to transferable embryos p−: small follicles not associated to transferable embryos, g−: large follicles not associated to transferable embryos.

### DETAILED DESCRIPTION OF PREFERRED EMBODIMENTS

The present invention provides, by the analysis of marker expression, methods of assessing maturity of a mammalian ovarian follicle and methods to optimize assisted reproduction techniques to obtain follicles at a “ideal” stage of maturity for sampling competent oocytes. Competent oocytes are more likely to experience successful fertilization and the resulting embryos are more likely to be of “better” or “high” quality (e.g. viability, likelihood of successful implantation, resistance to long-term storage and freezing, etc).

The invention identifies biological ovarian markers from the follicular cells (and follicular fluid), the cumulus cells and from oocytes which are indicative of follicular maturity in mammals.

### DEFINITIONS

For the purpose of the present invention the following terms are defined below.

As used herein and in the appended claims, the singular forms “a”, “an”, and “the” include plural referents unless the context clearly indicates otherwise. Thus, for example, reference to “a marker” includes one or more of such markers and reference to “the method” includes reference to equivalent steps and methods known to those of ordinary skill in the art that could be modified or substituted for the methods described herein.

As used herein the term “assisted reproduction” or “AR” broadly refers to methods, procedures and techniques wherein oocytes and/or embryos are manipulated, including, but not limited to, in vitro fertilization (IVF), in vitro maturation (IVM), artificial insemination (Al), intracytoplasmic sperm injection (ICSI), zygote intrafallopian transfer (ZIFT), pronuclear stage tubal transfer (FROST), and embryo transfer.

The term “subject” includes living organisms in which evaluation of follicular maturity is desirable. The term “subject” includes female animals (e.g., mammals (e.g., cats, dogs, horses, pigs, cows, goats, sheep, rodents (e.g., mice or rats), rabbits, squirrels, bears, primates (e.g., chimpanzees, monkeys, gorillas, and humans)), as well as avian (e.g. chickens, ducks, Peking ducks, geese), and transgenic species thereof. Preferably, the subject is a mammal. More preferably, the subject is a human. Even more preferably, the subject is a human patient in need of or receiving in vitro fertilization treatment and more particularly, a human patient subjected to control ovarian stimulation (COS).

The term “control ovarian stimulation” or “COS” as used herein refers to the process where a regimen of fertility medications is used to stimulate the development of multiple follicles of the ovaries in one single cycle, resulting in release of a larger-than-normal number of oocytes (e.g. “superovulation” or “ovarian hyperstimulation”).

The term “ovarian marker” as used herein refers to particular genes expressed in an ovarian follicle and which expression is indicative of the maturity of that particular follicle. As used herein, genes expressed in an ovarian follicle include genes from cumulus cells, follicular cells and from the oocyte comprised in the ovarian follicle.

The term “follicular maturity” or “maturity of a follicle” as used herein is intended to mean a stage of development or readiness of a follicle to generate a competent oocyte. Before such stage the follicle is qualified as immature and after this stage as overmature.

The term “competent”, “competence” or “competency” as used herein is intended to refer to the competence, or competency, both terms being equivalent, of an oocyte for fertilization, implantation and development into living individual. For the purpose of clarification, oocyte competency is directly dependent upon follicular maturity and competence will be lower if the follicle is immature or overmature.

The term “cumulus cells” refers to cells which originates from or are connected to (e.g. surrounding and nourishing) the oocyte in an ovarian follicle. This cluster of cells is also termed the cumulus oophorus.

The term “follicular fluid” refers to the liquid which fills the follicular antrum and surrounds the ovum (oocyte) in an ovarian follicle. The term “follicular cells” as used herein defines the cells that are obtained by follicular aspiration at the time of oocyte collection, these cells consisting mainly of granulosa cells and cumulus cells. When the antrum develops and enlarges, the follicular cells divide into two functional groups: the cells in immediate contact with the oocyte which are called the cumulus cells (cumulus oophorus) and the mural granulosa cells which line the follicular wall around the follicular antrum. Cumulus cells express characteristics distinct from the mural granulosa cells. Those skilled in the art are aware that by aspirating follicular content near ovulation often result in a mix of cumulus and granulosa cells, and may be some blood. Since the most of the cumulus cells are removed with the oocyte, the follicular cells remaining for the analysis are mainly granulosa cells. Because the follicular fluid comprises proteins and other factors produced or secreted by the follicular cells, expression of certain follicular cells markers according to the invention can be assessed indirectly by assessing expression of the corresponding polypeptide in the follicular fluid. Therefore, “follicular cells” encompasses “follicular fluid” where meaningful and appropriate.

An “oligonucleotide” or “polynucleotide” is a nucleic acid molecule ranging from at least 2, preferably at least 8, 15 or 25 nucleotides in length, but may be up to 50, 100, 1000, or 5000 nucleotides long or a compound that specifically hybridizes to a polynucleotide. Polynucleotides include DNA and fragments thereof, RNA and fragments thereof, cDNAs and fragments thereof, expressed sequence tags, artificial sequences including randomized artificial sequences.

As used herein, the term “polypeptide” or “protein” refers to any amino acid sequence derived from the expression of a nucleic acid sequence or gene encoding an ovarian marker as defined herein. The term is intended to encompass complete proteins and fragments thereof.

The term “transferable embryo” refer to an embryo (cryopreserved or not) that is suitable for transfer (with or without prior cryopreservation) in a human fertility clinical environment. In some embodiments, the term refers to an embryo at development stage ranging from morulae to blastocyst. The term transferable embryo is used in opposition to the term “not transferable embryo” which refers to an embryo with arrested development to a stage from 1 pronucleus to 10 cells.

The term “small follicle” refers to follicles associated to about 0.5 to about 1.5 ml of follicular fluid, which is equivalent to about 10 to about 14 mm in sphere diameter. The follicle diameter can be measured by ultrasonography before transvaginal punction.

The term “medium size follicle” refers to follicles associated to about 2 to about 3 ml of follicular fluid, which is equivalent to about 15 to about 18 mm in sphere diameter. The follicle diameter can be measured by ultrasonography before transvaginal punction

The term “large follicle” refers to follicles associated to about 3.5 to about 5 ml of follicular fluid, which is equivalent to 19 to 21 mm in sphere diameter. The follicle diameter can be measured by ultrasonography before transvaginal punction.

### Evaluation of Follicular Maturity

Evaluation of follicular maturity (and associated capacity of mature follicles to generate competent oocytes) may serves different uses. For instance, in one embodiment evaluation of follicular maturity is carried out in the course of optimization of assisted reproduction (AR) techniques (e.g. in vitro fertilization (IVF), artificial insemination (Al), intracytoplasmic sperm injection (ICSI), zygote intrafallopian transfer (ZIFT), pronuclear stage tubal transfer (PROST), and embryo transfer).

Therefore, the markers of the invention may be used to assess and/or to optimize methods for ovarian stimulation and/or for modifying or optimizing an in vitro maturation medium (e.g. identity and/or levels of components) and/or for modifying or optimizing in vitro maturation conditions (e.g. temperature, incubation time period with certain compounds, etc). Accordingly, a related aspect of the invention concerns methods for optimizing assisted reproduction techniques in a human subject, including but not limited to a method for optimizing in vitro maturation (IVM)) as defined hereinbefore.

The markers of the invention may be used to evaluate follicular maturity status in the course of treatment of COS, including in situations where COS is administered to women suffering from polycystic ovarian syndrome (PCO) or hyper stimulation syndrome (HSS), or women suffering from any similar reproductive disorder. Knowing follicular maturity status of ovarian follicles from such women will help in optimizing the COS protocols and maximize likelihood of pregnancy.

The assessment of marker expression in follicular cells (e.g. granulosa cells, cumulus cells) and/or oocytes according to the invention may also be useful to assist the proper function of affected gene expression pathways for example, assay the effects of toxicants on human reproduction (e.g. formation and development of human oocytes and/or human embryos).

Another related aspect concerns methods wherein assessment of the expression of the biological markers of the invention are used to determine the suitability of a female individual for assisted reproduction treatment, and/or for optimizing for ovarian stimulation protocols.

In some embodiments, the method for assessing maturity of a mammalian ovarian follicle further comprises selecting for in vitro fertilization (IVF) and/or in vitro maturation (IVM) oocytes originating from follicles having a desired maturity. Therefore, the markers according to the invention may be useful for optimizing selection of competent oocytes and optimize obtaining fertilized oocytes and embryos capable to implant (or, more accurately, successfully implant) in the uterus of a recipient female and to develop into a living being. Accordingly, the markers and methods of the invention may be useful to perform the screening of competent embryos before their transfer in a recipient human or animal female. Yet, the oocyte, the follicular cells, and/or cumulus and granulosa cells markers may be used for evaluating whether a female subject is fertile or infertile.

In some embodiments, evaluation of follicular maturity and selection of oocytes originating from follicles having a desired maturity is performed before fertilization, to assist selecting competent oocytes, to assist in maximizing the generation of chromosomally normal embryos or to assist in minimizing the generation of chromosomally abnormal embryos. Yet, in another embodiment, the follicular fluid, the cumulus cells and/or granulosa cells markers are used to assess whether an oocyte is chromosomally normal (e.g. in vitro assessment of oocyte aneuploidy). In another embodiment evaluation of follicular maturity and selection of oocytes originating from follicles having a desired maturity is performed before implantation to assist in maximizing the implantation of chromosomally normal embryos or to assist in minimizing the implantation of chromosomally abnormal embryos (e.g. diagnose chromosome abnormality).

One particular aspect of the invention concerns an in vivo method for assessing a compound with stimulatory or inhibitory activity to follicular maturation in a subject, the method comprising the steps of:
contacting an oocyte and/or follicular cell(s) (e.g. granulosa cells, cumulus cells) with a compound to be screened for activity to stimulate or inhibit follicular maturation;
determining an expression level of at least one ovarian marker in the oocyte and/or follicular cell(s) contacted with said compound, wherein the at least one ovarian marker is selected from the group consisting of the genes listed in Tables I, II and III, and combinations thereof;
comparing the expression level measured with the expression level of non-contacted oocyte(s) and/or follicular cell(s);
   wherein a difference in these expression levels is indicative of the compound stimulatory or inhibitory effect.

In practice, evaluating maturity of an ovarian follicle is carried out by assessing expression of one or the biological marker(s) according to the invention from the same follicle from which are sampled the follicular cell(s) and/or the oocyte. In preferred embodiments, the subject's follicular cell(s) and/or oocyte is(are) obtained before ovulation by aspirating said ovarian follicle comprising said follicular cell(s), cumulus cell(s) and/or oocyte. In particular embodiments the follicular cell(s) is(are) granulosa cell(s) or cumulus cell(s).

Preferably, the follicles, follicular cells oocytes are human. However, the follicles, follicular cells and/or oocytes may be obtained from other non-human animals, for instance domesticated animals such as cows.

Quantity of fluid or number of cells (one or more) to be used for assessing expression levels will vary according to various factors, including but not limited to the particular marker being assessed, the source and quality of the sample, the measurement technique being used, the subject's condition, the collection protocol in the clinic, etc.

According to the present invention, the follicles, follicular cells and/or oocytes can be harvested by methods and techniques known in the art, including direct aspiration of the ovarian follicle of a subject with an appropriate needle via the subject's vagina or any other suitable route. Is some embodiment, follicular cells and/or oocytes may be obtained by puncture of an ovarian follicle from an ovary outside the subject's body.

It is also conceivable according to the invention to assess indirectly expression of selected markers by measuring culture medium in which the follicular cells and/or oocytes are or have been cultured. The present invention encompasses in vitro, in vivo and ex vivo follicles. The uses of metabolomic approaches are also within the scope of the invention.

### Measurement Methods

The invention contemplates using methods known to those skilled in the art for the identification of differently expressed markers and/or assessment of markers expression levels or marker expression products, such as RNA and protein, in follicular fluid, cumulus cells, and follicular cells. As used herein, the term “marker expression” or “expression of a [α] marker” encompasses the transcription, translation, post-translation modification, and phenotypic manifestation of a gene, including all aspects of the transformation of information encoded in a gene into RNA or protein. By way of non-limiting example, marker expression includes transcription into messenger RNA (mRNA) and translation into protein.

The terms “assessing expression” is meant an assessment of the degree of expression of a marker in a sample at the nucleic acid or protein level, using technology available to the skilled artisan to detect a sufficient portion of any marker expression product (including nucleic acids and proteins) of any one of the genes listed herein in Tables I, II and III and/or any of the sequences listed herein in the accompanying sequence listing, such that the sufficient portion of the marker expression product detected is indicative of the expression of any one of the genes listed herein in Tables I, II and III and/or any one of the sequences listed herein in the accompanying sequence listing.

Any suitable method known in the art can be used to measure the marker's expression. For instance, assessment of the expression of the markers according to the invention may comprise detecting and/or measuring the level of one or more marker expression products, such as mRNA and protein.

According to the invention, specific markers are selected depending of the origin of the biological materials. For instance, in one embodiment the follicular cell marker is a granulosa cell marker which is selected from the group of genes listed in Table I and combinations thereof. In some embodiments, the invention comprises assessing expression of granulosa cells marker(s) by measuring levels of expression at the polynucleotide level. In some embodiments, the invention comprises assessing expression of follicular cell marker(s) by measuring levels of expression at the polypeptide level, including but not limited to measuring levels of entire proteins, polypeptides, and fragments of the polypeptides encoded by the polynucleotides. Polynucleotide and polypeptide sequences of the granulosa cell markers according to the invention can easily be found by consulting the NCBI database for the GeneID numbers provided in Table I.

In another embodiment the follicular cell marker is a cumulus cell marker which is selected from the genes listed in Table II and combinations thereof. In some embodiments, the invention comprises assessing expression of cumulus cell marker(s) by measuring levels of expression at the polynucleotide level. In some embodiments, the invention comprises assessing expression of cumulus cell marker(s) by measuring levels of expression at the polypeptide level, including but not limited to measuring levels of entire proteins, polypeptides, and fragments of the polypeptides encoded by the polynucleotides. Polynucleotide and polypeptide sequences of these genes can easily be found by consulting the NCBI database for the GeneID numbers provided in Table II.

Yet, in another embodiment the marker is a oocyte marker which is selected from the genes listed in Table III and combinations thereof. In some embodiments, the invention comprises assessing expression of oocyte marker(s) by measuring levels of expression at the polynucleotide level. In some embodiments, the invention comprises assessing expression of oocyte marker(s) by measuring levels of expression at the polypeptide level, including but not limited to measuring levels of entire proteins, polypeptides, and fragments of the polypeptides encoded by the polynucleotides. Polynucleotide and polypeptide sequences of these genes can easily be found by consulting the NCBI database for the GeneID numbers provided in Table III.

Assessment of the expression of the ovarian markers described herein may comprises measuring polynucleotide levels (e.g. DNA and/or mRNA levels) and/or polypeptide expression levels for such markers. In some embodiments assessment of the marker's expression comprises measuring polynucleotide, or fragments thereof (e.g. 10, 25, 50, 75, 100, 150, 200, 250, 300, 400, 500 or more nucleotides in length), the polynucleotide comprising a sequence as set forth in NCBI for the GeneID numbers provided in Tables I, II and III. In other embodiments assessment of the marker's expression comprises measuring a polypeptide, or a fragment thereof (e.g. 10, 15, 25, 50, 75, 100 or more amino acid in length), the polypeptide comprising an amino acid sequence as set forth in NCBI for the GeneID numbers provided in Tables I, II and III. Those skilled in the art will know how to select appropriate markers reported herein and identify suitable polynucleotide or polypeptide sequences providing a desired sensitivity and specificity. In particular embodiments, the ovarian marker is an individual marker selected from PDE8B, THBD, TLR2, CHODL, TGFBR2 and combinations thereof comprising 2-5 of these markers.

In some embodiments, assessment of the marker's expression is carried out by using genetic tools and related molecular biology techniques. Any conventional technique of molecular biology known to those in the art can be used, including but not limited to amplification and hybridization-related methods, and more particularly nucleic acid arrays and microarrays, PCR amplification, ligase chain reaction (LCR), polynucleotide hybridization assays (e.g. Northern blot, Southern blot, etc.), deep sequencing and the like. Those skilled the art are capable of selecting suitable tools and techniques for measurement methods of gene expression.

In some embodiments, the invention contemplates the use of nucleic acid probes capable of specifically hybridizing to a mRNA of interest, and oligonucleotides or PCR primers capable of specifically amplifying a target nucleotide sequence. The nucleic acid probes, oligonucleotides or PCR primers may be of about 5 to 200 about nucleic acids in length (e.g. about 5, about 10, about 15, about 20, about 25, about 30, about 50, about 75, about 100, about 125, about 150, about 175, about 200, about 300, about 400, about 500). The ways of preparing such nucleic acid probes, oligonucleotides or PCR primers are well known by persons skilled in the art. PCR analysis is preferably performed as reverse-transcriptase PCR(RT-PCR). PCR amplification products can be measured in real time for precise quantification (Real-time PCR).

Hybridized nucleotides can be detected by detecting one or more labels attached to sample nucleic acids or to a probe. Labels and dyes can also be used for protein and polypeptide detection. Examples of useful labels for use in the present invention include, but is not limited to, biotin for staining with labeled streptavidin conjugate, anti-biotin antibodies, magnetic beads, fluorescent dyes (e.g. fluorescein, texas red, rhodamine, green fluorescent protein, and the like), radiolabels, phosphorescent labels, enzymes (e.g. horse radish peroxidase, alkaline phosphatase), and colorimetric labels such as colloidal gold or colored glass or plastic.

In some embodiments, assessment of the marker's expression is carried out by using polypeptide-related tools and detection techniques. Any conventional technique known to those in the art can be used, including but not limited to competitive and non-competitive immunoassays (e.g. sandwich assays, ELISA, RIA, chemiluminescent detection, etc.), electrophoresis and chromatography (liquid chromatography, capillary electrophoresis, quantitative western blotting, etc.), fluorescent probes, absorption matrices, mass spectrometry, and the like. Antibodies capable of specifically binding to polypeptides expressed by the gene of interest may be particularly useful. In addition, any established or newly quantitative technique known in the art can be used, alone or in combination with other techniques, in the accurate assessment of follicular cells, cumulus cells or oocyte markers expression. Those skilled the art are capable of selecting suitable tools and techniques for measurement methods of polypeptide expression levels.

The present invention may also make use of various computer program products and software for a variety of purposes, such as probe design, management of data, statistical analysis, mathematical algorithms and instrument operation. Additionally, the present invention may have include methods for providing results and genetic information over networks such as the Internet.

In another embodiment, the maturity of the follicle can be addressed by the measurement of a plurality of follicular cells and/or oocyte markers according to the invention. Measurement of a plurality of markers may be helpful in drawing gene expression profile pattern of a tested follicle and in establishing a subject's expression profile. An expression profiles may be helpful in establishing more finely the maturity of the follicle as defined herein. In some embodiments, the methods of the invention comprises assessing expression of at least 2, 3, 4, 5, 6, 7, 8, 9, 10 or more granulosa cell markers. In some embodiments, the methods of the invention comprises assessing expression of at least 2, 3, 4, 5, 6, 7, 8, 9, 10 or more cumulus cell markers. In some embodiments, the methods of the invention comprises assessing expression of at least 2, 3, 4, 5, 6, 7, 8, 9, 10 or more oocyte markers. In some embodiments, the methods of the invention comprises assessing expression of at least 2, 3, 4, 5, 6, 7, 8, 9, 10 or more markers from different source (e.g. from oocyte(s) and from a granulosa cell(s), from an oocytes(s) and cumulus cell(s) and/or granulosa cell(s) and cumulus cell(s)).

According to particular embodiments, the methods of the invention comprises assessing expression a combination of at least two granulosa cell markers, the combination being selected according to Table A hereinafter.

According to particular embodiments, the methods of the invention comprises assessing expression a combination of at least three granulosa cell markers, the combination being selected according to Table B hereinafter.

According to particular embodiments, the methods of the invention comprises assessing expression a combination of at least four granulosa cell markers, the combination being selected according to Table C hereinafter.

According to particular embodiments, the methods of the invention comprises assessing expression a combination of at least two markers from different source (e.g. follicular fluid, cumulus cell and/or follicular cells).

Similarly, the assessment of the expression of one or more follicular cells or oocyte markers according to the invention can be used in combination with any other suitable indicator of follicular maturity, with any other suitable indicator oocyte competency, with any other suitable indicator of a female subject fertility or infertility, with any other suitable indicator of an oocyte chromosomal defectiveness, etc. in a subject. Examples of possibly useful indicators include, but are not limited to, follicle size and volume, ratio of number/proportion of follicles, the age, body weight, general health, hormone levels (e.g. FSH, LH, AMH (Anti-Mullerian Hormone)), the time of the menstrual cycle, and hormonal treatment used.

TABLE A
Combination of at least two granulosa cell markers

| | PDE8B | THBD | TLR2 | CHODL |
|---|---|---|---|---|
| PDE8B | | | | |
| THBD | X | | | |
| TLR2 | X | X | | |
| CHODL | X | X | X | |
| TGFBR2 | X | X | X | X |

TABLE B
Combination of at least three granulosa cell markers

| | PDE8B & | PDE8B & | PDE8B & | THBD & | THBD & | TLR2 & |
|---|---|---|---|---|---|---|
| Gene | THBD | TLR2 | CHODL | TLR2 | CHODL | CHODL |
| PDE8B | | | | | | |
| THBD | | | | | | |
| TLR2 | X | | | | | |
| CHODL | X | X | | X | | |
| TGFBR2 | X | X | X | X | X | X |

TABLE C
Combination of at least four granulosa cell markers

| | PDE8B & | PDE8B & | THBD & |
|---|---|---|---|
| | THBD & | TLR2 & | TLR2 & |
| Gene | TLR2 | CHODL | CHODL |
| CHODL | X | | |
| TGFBR2 | X | X | X |

### Control Expression Level

In some embodiment, the methods of the invention further comprise comparing the expression level of the biological marker with a control expression level in control follicular cell(s), cumulus cell(s) or oocyte(s). As used herein, “control expression level” is meant any value, including a predetermined value or a range of values, that is used for purposes of comparison. A control expression level can reflect the outcome of a single experiment or assay, or it can be a statistical function of multiple experiments or assays. A control expression level can also reflect the presence or absence of a signal. A control expression level can be generated from a prior measurement from the same subject or a measurement from a sample (e.g. follicular cells, cumulus cells or oocyte) from a single or from a pool of two or more mature follicles whose maturity as been evaluated using any other suitable indicator of follicular maturity as defined above, including for instance the capability for that follicle to provide an oocyte competent or not for fertilization or capability to provide an oocyte competent or not for embryo development.

Comparing the expression level of the biological marker with a control expression level may comprise comparing two values (or a set of values) in parallel, or comprise calculating a difference (e.g. a threshold level) or calculating a ratio in expression level(s). Such comparison may provide an absolute or relative gene/peptide expression. Whenever necessary, it is also possible to normalize the measured marker levels using available normalization tools, including using level of expression of the biological marker over level of expression of a housekeeping gene, including but not limited to ACTB (Beta actin), GAPDH (glyceraldehyde-3-phophate dehydrogenase), PPHLN1 (Periphilin 1), PPIA (cyclophylin A) and 18S ribosomal RNA (18S).

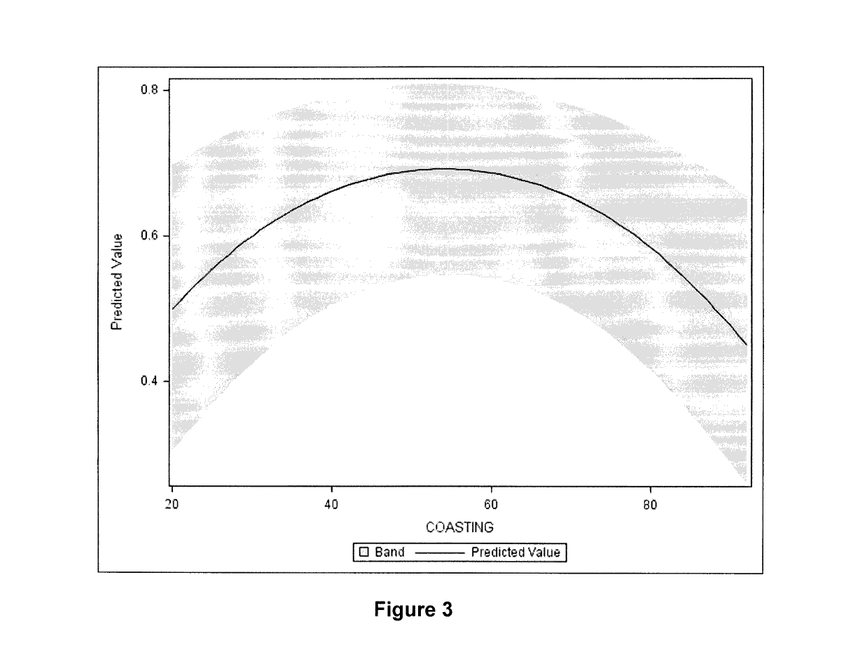

According to some embodiments, when expression level of a marker in a tested follicular cell(s), cumulus cell(s) or oocyte(s) is lower than the average level of the same marker from the follicular cell(s), cumulus cell(s) or oocyte(s) originating from a group of mature follicles, it is deemed immature. On the contrary, a tested follicular cell(s), cumulus cell(s) or oocyte(s) having an expression level of a marker similar or greater than the expression levels in the controls (mature group) will indicate that the follicle is mature. If the levels of expression are too high compared to the controls (mature group), the follicle may be overmature. Under such circumstances, the ratio of the expression level of a marker in a tested follicle over the expression level of a marker in a control follicle can be from about 1.5 above control to 150 (e.g. above 2, above 5, above 10, above 25, above 50, above 75, above 100 or more) and preferably above 2 for an follicle to be deemed mature. However, it is understood that for some markers, it may be the opposite, i.e. a lower expression level of an ovarian marker in a tested follicular cell(s), cumulus cell(s) or oocyte(s), when compared to appropriate controls (mature group) will indicate that the oocyte is mature or overly mature and a higher expression level will indicate that the oocyte is mature or immature. Those skilled in the art will be able, when considering the instant disclosure to determine whether it is a higher or lower expression of the ovarian marker which is indicative of higher or lower competency and calculate appropriate ratios. For instance, as illustrated in FIG. 3, the correlation between maturity of the follicle and the level of expression of the ovarian maker, may not necessarily be linear but may be parabolic.

Those skilled in the art also understand that average expression level of one or more selected markers may be preferable to select or to assess follicular maturity, and more particularly follicles providing mature oocytes likely to implant and to develop properly in the uterus up until the birth. For instance, in the case where the expression level of a marker in follicular cells, cumulus cells or oocyte of a tested follicle is within the range associated with expression levels of mature follicle (e.g. higher expression level compared to the range of immature follicle) the tested follicle will be deemed mature. On the contrary, if the level is below a defined or relative threshold (or above, depending on the particular marker) then the follicle will be considered immature or considered of lower potential. However, for some markers, the correlation may be inverter (i.e. lower level of expression of the marker reflecting an overmaturity.

### Induction of Follicular Maturity

Another aspect of the present invention relates to a method for inducing or improving follicular maturity. The method includes treating a subject with one or more factors known to modulate the expression of one or more selected follicular cells and/or oocyte markers according to the invention. The factor(s) is selected according to the markers and type of modulation that is desired (e.g. higher or lower levels of expression). For instance, administering a given hormonal treatment or a given schedule of treatment or a combination of dose and products (like FSH and LH) may increase (or reduce) the presence of markers and hence improve the maturity of the resulting follicles.

The ovarian markers of follicular maturity according to the invention may also be useful to validate treatments aimed as contraceptive. For instance, if higher levels of a given marker is indicative of better chances of pregnancy, a lower level would indicate a lower chance of pregnancy. Therefore treatments aiming at reducing the presence of such a marker could be developed for contraceptive purposes. Methods of decreasing gene expression can be applied through various hormonal treatments or direct signaling path with specific chemicals such as phosphodiesterase inhibitors (e.g. Viagra™) or through RNAi or synthetic oligomer.

### Drug Screening

A further aspect of the present invention relates a method for screening candidate compounds capable of increasing or decreasing the expression of markers of the invention as described herein. For example, but not limited to, isolated cumulus or granulosa cells put under in vitro culture conditions can be submitted to treatment with candidate compounds, and then tested for measuring the increase or decrease of expression levels of follicular maturity markers, therefore reflecting the effect of the candidate compound. This approach will allow the screening of compounds stimulatory or inhibitory to follicular maturation. The same compound testing can be performed under in vivo conditions, for instance following administration of a candidate compounds to subject, through which ovarian stimulation conditions can be tested for assessing expression of granulosa cells, cumulus cells or oocyte markers according to the invention, and/or for assessing the production of mature follicles.

According to a particular embodiment, the method for screening a compound stimulatory or inhibitory to mammalian follicular maturation comprises the steps of:
a) contacting granulosa cell(s) with a compound to be screened for activity to stimulate or inhibit follicular maturation;
b) determining an expression level of at least one granulosa cell marker in granulosa cells contacted with said compound, wherein said at least one granulososa cell marker is selected from the group consisting of the genes listed in Table I and combinations thereof;
c) comparing the expression level measured in step b) with the expression level of non-contacted follicular cells;
   wherein a difference in the expression levels is indicative of the compound stimulatory or inhibitory effect.

According to another embodiment, the method for screening a compound stimulatory or inhibitory to mammalian oocyte competence comprises the steps of:
a) contacting cumulus cell(s) with a compound to be screened for activity to stimulate or inhibit follicular maturation;
b) determining an expression level of at least one cumulus cell marker contacted with the compound, wherein the at least one cumulus cell marker is selected from the group consisting of consisting of genes listed in Table II and combinations thereof;
c) comparing the expression level measured in step b) with the expression level of non-contacted cumulus cells;
   wherein a difference in the expression levels is indicative of the compound stimulatory or inhibitory effect.

According to a further embodiment, the method for screening a compound stimulatory or inhibitory to mammalian follicular maturation comprises the steps of:
a) contacting an oocyte with a compound to be screened for activity to stimulate or inhibit follicular maturation;
b) determining an expression level of at least one oocyte marker in an oocyte contacted with the compound or in culture media deriving therefrom, wherein the at least one follicular cell marker is selected from the group consisting of the genes listed in Table III and combinations thereof;
c) comparing the expression level measured in step b) with the expression level of non-contacted oocyte(s);
   wherein a difference in said expression levels is indicative of the compound stimulatory or inhibitory effect.

### Kits and Arrays

A further aspect of the invention relates to a solid support and to kits. The solid supports and/or kits of the invention may be useful for the practice of the methods of the invention, particularly for diagnostic applications in humans according to the evaluation methods described hereinbefore.

A solid support according to the invention may comprise a compound for assessing expression of one or more follicular cells or oocyte markers as defined herein.

In one embodiment, the compound is a nucleic acid probe designed for specific detection of a marker according to the invention. The solid support may me a tube, a chip (see for instance Affymetrix GeneChip® technology), a membrane, a glass support, a filter, a tissue culture dish, a polymeric material, a bead, a silica support, etc. The invention also encompasses the use of techniques and tools relating to microfluidic and lab-on-chip technology.

In some embodiment the solid support is a nucleic acid array. Nucleic acid arrays that are useful in the present invention include arrays such as those commercially available from Affymetrix (Santa Clara, Calif.), Applied Biosystems (Foster City, Calif.) and from Agilent Technologies (Santa Clara, Calif.). Preferred arrays according to the invention typically comprises a plurality of different nucleic acid probes (e.g. a probes capable of hybridization with a follicular fluid, cumulus cell or granulosa cell markers as defined herein) that are coupled to a surface of a substrate in different, known locations. The array may be designed to detect sequences from an entire genome, or from one or more regions of a genome, for example selected regions of a genome such as those encoding for a protein or RNA of interest. Arrays according to the invention can be directed to a variety of purposes, including genotyping, diagnostics, mutation analysis, and marker expression. Arrays, also described as “microarrays” or “chips” may be produced and packaged using a variety of techniques known in the art.

According to a particular aspect, the invention relates to an array of nucleic acid probes immobilized on a solid support, the array comprising a plurality of probes hybridizing specifically to an ovarian marker associated with follicular maturity. The probes comprises a segment of at least twenty nucleotides exactly complementary to at least one reference sequence selected from the group of nucleic acid sequences encoding the genes listed in Tables I, II and III.

A kit of the invention may comprise at least one oligonucleotide hybridizing specifically with an ovarian marker associated with follicular maturity (i.e. an ovarian marker comprising a sequence selected nucleic acid sequences encoding the genes listed in Tables I, II and III). The kit may also comprise one or more additional components, such as a buffer for the homogenization of the biological sample(s), purified marker proteins (and/or a fragment thereof) to be used as controls, incubation buffer(s), substrate and assay buffer(s), standards, detection materials (e.g. antibodies, fluorescein-labelled derivatives, luminogenic substrates, detection solutions, scintillation counting fluid, etc.), laboratory supplies (e.g. desalting column, reaction tubes or microplates (e.g. 96- or 384-well plates), a user manual or instructions, etc. Preferably, the kit and methods of the invention are configured such as to permit a quantitative detection or measurement of the protein(s) or polynucleotide(s) of interest.

For instance, the kits may comprise at least one oligonucleotide which specifically hybridizes with nucleic acid molecules encoding any of the follicular cells, cumulus cells or oocyte markers defined herein, reaction buffers, and instructional material. Optionally, the at least one oligonucleotide contains a detectable tag. Certain kits may contain two such oligonucleotides, which serve as primers to amplify at least part of the markers. Some kits may contain a pair of oligonucleotides for detecting pre-characterized mutations in the oocyte, follicular fluid, cumulus cell or granulosa cell markers defined herein. Alternatively, the kit may comprise primers for amplifying at least part of the markers to allow for sequencing and identification of mutant nucleic acid molecules. The kits of the invention may also contain components of the amplification system, including PCR reaction materials such as buffers and a thermostable polymerase. In other embodiments, the kit of the present invention can be used in conjunction with commercially available amplification kits, such as may be obtained from GIBCO BRL (Gaithersburg, Md.) Stratagene (La Jolla, Calif.), Invitrogen (San Diego, Calif.), Molecular Devices (Sunnyvale, Calif.). The kits may optionally include instructional material, positive or negative control reactions, templates, or markers, molecular weight size markers for gel electrophoresis, and the like.

Kits of the instant invention may also comprise antibodies immunologically specific for follicular fluid, cumulus cell or granulosa cell markers defined herein and/or mutants thereof and instructional material. Optionally, the antibody contains a detectable tag. The kits may optionally include buffers for forming the immunocomplexes, agents for detecting the immunocomplexes, instructional material, solid supports, positive or negative control samples, molecular weight size markers for gel electrophoresis, and the like.

Those skilled in the art will recognize, or be able to ascertain using no more than routine experimentation, numerous equivalents to the specific procedures, embodiments, and examples described herein. Such equivalents are considered to be within the scope of this invention and covered by the claims appended hereto. The invention is further illustrated by the following examples, which should not be construed as further limiting.

### Example 1

### FSH Withdrawal in the Presence of Basal LH Results in Improved Developmental Competence of Oocytes in the Bovine Model

### Materials and Methods

### Chemicals

All reagents and media supplements used in these experiments were of tissue-culture grade and were obtained from Sigma Chemicals Co. (St. Louis, Mo.) unless otherwise indicated.

Ovarian Stimulation Treatment and Oocyte Recovery from Superovulated Animals

Each animal (6 commercial milking cycling Bos taurus Holstein cows) was exposed to the 4 conditions with at least one complete regular sexual cycle between 2 treatments and served as its own control. Each animal was treated during luteal phase to prevent spontaneous ovulation. The dominant follicle was aspirated 36 hours before administration of hormones. Cows were stimulated for 3 days with FSH (6×40 mg NIH Folltropin-V™, Bioniche Animal Health, Belleville, ON, Canada, given at 12-hour intervals), followed by a coasting (no FSH) period of four different durations (20, 44, 68, or 92 hours). Using transvaginal ultrasonography, follicular diameters were measured and cumulus-oocyte complexes (COCs) were collected by transvaginal puncture (PTV), under epidural, with a 18G needle and COOK aspiration unit (COOK Medical, Bloomington, Ind.). COCs and granulosa cells were collected in warm HEPES-buffered Tyrode's medium (TLH) containing Hepalean (10 UI/ml) and transferred to lab for in vitro maturation (half of the COCs) or treated (oocytes, cumulus, granulosa cells) for further studies.

### In Vitro Maturation

The COCs were placed in HEPES-buffered Tyrode's medium (TLH) solution (supplemented with 10% bovine serum, 0.2 mM pyruvate and 50 μg/ml gentamycin) and washed three times to remove follicular fluid. Healthy COCs were placed in droplets of maturation medium under embryo-tested mineral oil (#8410, Sigma). Maturation medium was composed of TCM199™ (Gibco 11150-059, Invitrogen, Burlington, ON, Canada), 10% FBS, 0.2 mM pyruvate, 50 μg/ml gentamycin, 5 μg/ml FSH and 0.5 μg/ml LH and 1 μg/ml estradiol. Maturation droplets were incubated for 24 hours at 38.5° C. with 5% CO₂, in maximal humidity.

### In Vitro Fertilization

After 24 hours of in vitro maturation, COCs were collected and washed twice in TLH medium before being transferred in groups of 5 into 48-μl droplets under mineral oil. The droplets consisted of modified Tyrode lactate medium (TL) supplemented with 0.6% (w/v) free fatty acid BSA, 0.2 mM pyruvic acid, 2 μg/ml heparin and 50 μg/ml gentamycin. Oocytes were transferred 15 min prior to semen addition and 2 μl of PHE (1 mM hypotaurine, 2 mM penicillamine, 250 mM epinephrine) were added to each droplet to stimulate sperm motility. The same semen was used for each in vitro fertilization (IVF) (Centre d'Insemination Artificielle du Québec; CIAQ, St-Hyacinthe, QC, Canada). The spermatozoa previously stored in liquid nitrogen were thawed for 1 min in 35° C. water, added to a discontinuous Percoll™ gradient (45% over 90% Percoll™ [Sigma-Aldrich]), and centrifuged at 700×g for 30 min at 26° C. The supernatant containing the cryoprotectant and the dead spermatozoa was discarded, and the pellet was then resuspended in 1 ml of modified TL and centrifuged at 250×g for 5 min at 26° C. The resuspended spermatozoa were counted on a hemocytometer and diluted with IVF medium to obtain a final concentration of 1×10⁶ cells/ml. Finally, 2 μl of the sperm suspension were added to the droplets containing the matured COCs. The fertilization medium was incubated at 38.5° C. for 15-18 hours in a humidified atmosphere of 95% air and 5% CO₂.

### In Vitro Culture

For culture, embryos were placed in groups of 10 in 10-μl droplets of modified synthetic oviduct fluid (mSOF) with non-essential amino acids, 3 μM EDTA and 0.4% fatty acid-free BSA (ICP-Bio, Auckland, New Zealand) under embryo-tested mineral oil (#8410, Sigma). The embryo culture dishes were incubated at 38.5° C. with 6.5% CO₂, 5% O₂ and 88.5% N₂ in 100% humidity. Embryos were transferred in new 10-μl droplets of mSOF containing non-essential and essential amino acids 72 hours post-fertilization and again 120 hours post-fertilization in 20-pi droplets of mSOF containing non-essential and essential amino acids to prevent toxicity due to ammonium concentration and nutrient depletion caused respectively by amino acid degradation and embryo metabolism. Blastocyst development was monitored at days 7 and 8 post-fertilization.

### Statistical Analysis

Statistical analyses were performed with SAS™ software. The dichotomic variable blastocyst stage followed a binomial distribution and was used in the General Estimating Equation model (GEE). The variable coasting period was considered firstly as a continuous variable. This permitted the regression model of the blastocyst stage data to be determined. The coasting period resulting in the maximal blastocyst probability was determined with the Delta method. Secondly, the coasting period was considered as a categorical variable and the different treatments were compared (chi-square). Follicle data followed a Poisson distribution and were used in log linear model to compare each coasting period within each follicle group, and each follicle group within each coasting period (chi-square).

### Results

### Developmental Competence According to the Coasting Period

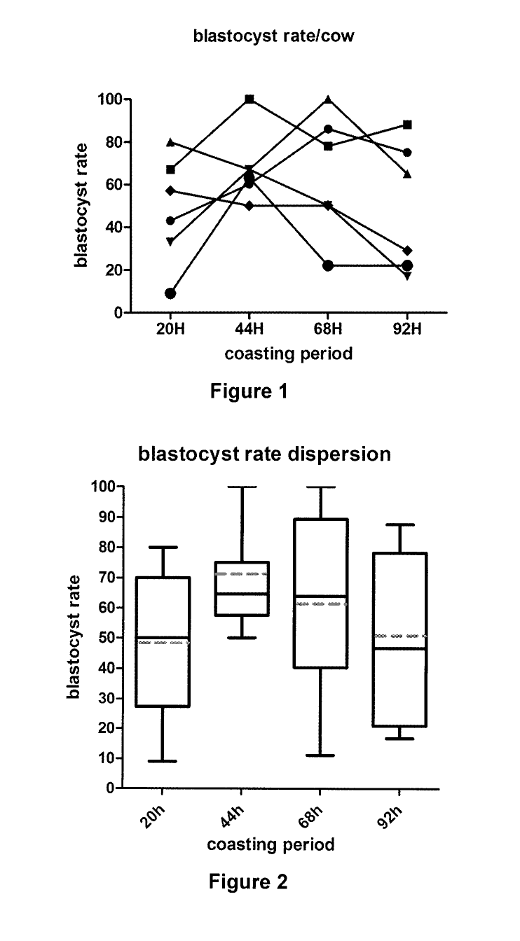

The impact of coasting on blastocyst rate is described in Table IV (absolute data) and FIG. 1. In this figure, each cow is presented individually to illustrate the variations amongst animals in relation to blastocyst developmental rates. The fact that each animal represents its own control adds to comparison values and supports the observation of the progressive effect of FSH withdrawal.

To better describe the overall effect of FSH, blastocyst rates were analyzed in box plots (FIG. 2). When oocytes were collected 20 hours after the last FSH injection, blastocyst rates varied from 9% to 80%. They varied from 50% to 100% at 44 hours, from 22% to 100% at 68 hours and finally from 22% to 88% at 92 hours. At 20, 44, 68, and 92 hours, the median and mean values were 50% and 49%, 65% and 71%, 64% and 61%, and 47% and 51%, respectively.

Considering the coasting period as a mathematical categorical variable, the probability of blastocyst production was estimated to be 48.9%, 70.6%, 63% and 46%, respectively, for the 4 coasting periods (20, 44, 68, and 92 hours). There were no difference between 20 and 92 hours, and between 44 and 68 hours. There was a tendency for a higher blastocyst rate at 44 hours than at 20 hours (p=0.063). There were significantly more blastocysts at 44 hours than at 92 hours (p=0.0199) as well as more blastocysts at 68 hours than at 20 (p=0.0129) or 92 hours (p=0.0004). Considering the coasting period as a mathematical continuum variable, a quadratic regression was calculated converging with the blastocyst data (FIG. 3). The equation of the blastocyst probability model is:

Log(p/1−p)=−1.2353+0.0757 coasting−0.0007 coasting×coasting.

Using this equation, the ideal FSH coasting to maximize blastocyst rate (69%) is 54.07+/−7.71 hours.

### Follicular Dynamics According to the Coasting Period

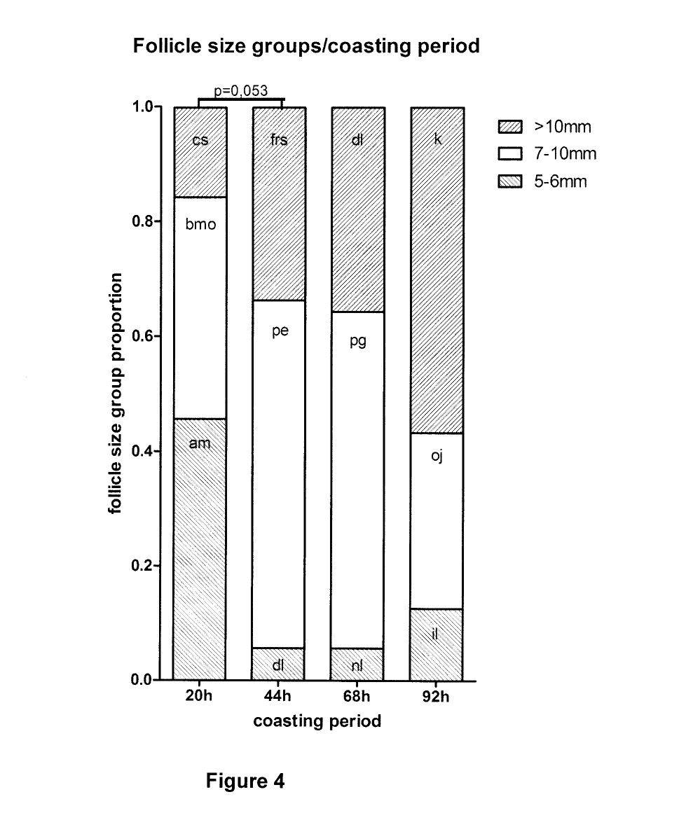

In our analysis of the follicular dynamics in this experiment, the follicles were classified in 3 groups (5-6 mm, 7-10 mm and above 10 mm). For the 5- to 6-mm follicles, there was significantly (p<0.0001) more follicles in this group at the earliest time (20 hours) of coasting compared with 44, 68, or 92 hours (FIG. 4 and Table V). There were less 7- to 10-mm follicles at 20 hours of coasting than at 44 (p=0.0047) and 68 hours (p=0.0243). Also, there were less 7- to 10-mm follicles at 92 hours than at 44 (p=0.0017) and 68 hours (p=0.0097). In the last group of follicles (>10 mm), there was no statistical difference between 44 and 68 hours. Finally, there were statistically more >10-mm follicles at 92 hours than at 20 (p<0.0001), 44 (p=0.0052), and 68 hours (p=0.0290).

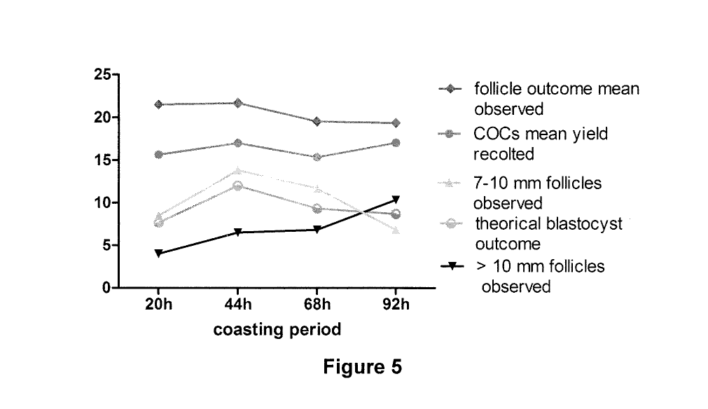

The other meaningful observation when the follicles were analyzed was that the overall number did not change after FSH withdrawal and remained at around 20 follicles per animal (over 5 mm) (FIG. 5). FIG. 5 also illustrates the shift from the 7-10 mm to the larger than 10 mm at the last coasting time (92 hours), indicating that the follicles were still growing. This increase in follicular size occurred at the same time as the observation of lower blastocyst rates from 68 to 92 hours.

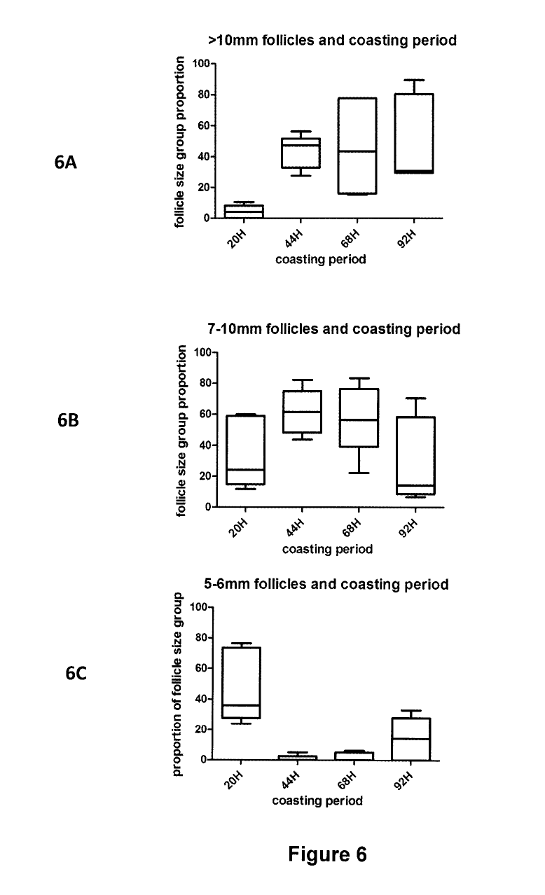

This change in follicular size prompted us to analyze the possible relationship between the number/proportion of follicles 7-10 mm in size and the blastocyst rate. The first obvious observation was that the blastocyst curve mimics the 7-10 mm curve as shown in FIG. 6B, while evolution of the proportion of larger follicles did not reflect the developmental outcome of enclosed oocytes. Considering the best blastocyst rate for each cow, the 7-10-mm follicles proportion was statistically positively correlated with blastocyst rate (R²=0.844, p=0.0096).

TABLE IV
Potential blastocyst yield per cow
Data are expressed as follows: number of blastocyst/half of COCs recovered (blastocyst rate).

| Coasting | | | | | | | |
|---|---|---|---|---|---|---|---|
| period | V1 | V2 | V3 | V4 | V5 | V6 | Total |
| 20 h | 3/7 (43%) | 6/9 (67%) | 8/10 (80%) | 1/3 (33%) | 4/7 (57%) | 1/11 (9%) | 23/47 (49.0%) |
| 44 h | 3/5 (60%) | 10/10 (100%) | 12/18 (67%) | 4/6 (67%) | 2/4 (50%) | 5/8 (63%) | 36/51 (71.0%) |
| 68 h | 6/7 (86%) | 7/9 (78%) | 7/7 (100%) | 4/8 (50%) | 3/6 (50%) | 1/9 (11%) | 28/46 (61.0%) |
| 92 h | 3/4 (75%) | 7/8 (88%) | 11/17 (65%) | 1/6 (17%) | 2/7 (29%) | 2/9 (22%) | 26/51 (51.0%) |
| Total | 15/23 (65%) | 30/36 (83%) | 38/52 (73%) | 10/23 (44%) | 11/24 (46%) | 9/37 (24%) | 113/195 (58.0%) |

TABLE V
P-values associated with follicle size group and coasting period in cow

| | | 5-6 mm | | | | 7-10 mm | | | |
|---|---|---|---|---|---|---|---|---|---|
| | | 20 | 44 | 68 | 92 | 20 | 44 | 68 | 92 |
| 5-6 mm | 20 | | ***<0.0001 | ***<0.0001 | ***<0.0001 | NS | | | |
| | 44 | | | NS | NS | | ***<0.0001 | | |
| | 68 | | | | NS | | | ***<0.0001 | |
| | 92 | | | | | | | | ***0.0003 |
| 7-10 mm | 20 | | | | | | **0.0047 | *0.0243 | NS |
| | 44 | | | | | | | NS | **0.0017 |
| | 68 | | | | | | | | **0.0097 |
| | 92 | | | | | | | | |
| >10 mm | 20 | | | | | | | | |
| | 44 | | | | | | | | |
| | 68 | | | | | | | | |
| | 92 | | | | | | | | |

NS: non-significant,
*p-value < 0.05,
**p-value < 0.01,
***p-value < 0.001

### Discussion

The main weaknesses of assisted reproduction technologies (ART) when applied to large mammalian species are a low success rate attributed to low oocyte competence, and a high degree of variability among individuals that could be associated to genetic and environmental factors. Using animals housed in the exact same conditions and applying the same exact treatments to each animal in a randomized scheme represents an important effort to minimize the impact of individuals and/or the environment on the ovarian response. In addition, for more relevance, the chosen animals were not heifers but lactating commercial cows to better reflect the population normally presenting fertility troubles and thus requiring infertility treatments.

In terms of blastocyst rate and follicle grouping, data dispersion was similar for all coasting periods (FIG. 1). This reflects the observed diversity in superovulation responses (Kafi, 1997, Mapletoft, 2002). In this group of 6 animals, when considering data dispersion, blastocyst rate and follicle groups, 44 hours of coasting was clearly the less variable condition tested. This result illustrates for the first time that some synchrony is possible throughout a cohort of follicles in mono-ovulating species.

Although follicles are usually and basically reported by their sizes, it has been a challenge to link follicle size and competence in several species. In cows, a higher number of blastocysts are obtained from follicles >6 mm than from 2-6 mm follicles (Lonergan et al., 1994), and from 2-8-mm follicles than from 1-2-mm ones (Pavlok et al., 1993). Individually cultured cumulus-oocyte complexes (COCs) originating from 3-5-mm and >5-mm follicles are linked to higher blastocyst rates (Blondin & Sirard, 1995). One must keep in mind that protocols are always different and that errors may occur during follicular fluid recovery. Nevertheless, in general, a linear positive correlation between follicular size and competence is observed, at least until the 8-mm follicle stage in cows. Surprisingly, this study indicated that the best blastocyst rate (44-68 hours) was not related to the largest cohort of follicles (92 hours), i.e., the follicle cohort including the higher number of >10-mm follicles. Statistically, the presumed largest follicles are still growing from 68 to 92 hours and the rate of blastocyst is lower at such time. Therefore, we can assume that, for unknown reasons, the correlation between size and competence is not positive in the largest follicles. Consistent with our hypothesis, Hagemann et al. (1999) did not obtain a better developmental rate with follicles >13 mm compared to 9-12-mm follicles.

The fact that growing follicles may contain compromised oocytes has been observed before. Two other different experimental conditions support such an observation. The first one was extending exposure to progesterone supplementation thereby preventing ovulation. It was associated with continuous follicle growth and reduced fertility (Sirois & Fortune, 1990; Bridges & Fortune, 2003). A second study used GnRH inhibition in order to obtain similar results. Increased atresia rate associated with decreased blastocyst rate were noted (Oussaid et al., 2000; van de Leemput et al., 2001). Although previous studies are consistent with our data supporting a link between largest follicles and a reduced blastocyst rate, it is not possible to rule out that decreased competence may be caused by smaller follicles as the oocytes were not evaluated according to follicular dimension (7-10 mm).

Follicles collected in an active growth phase have a low developmental competence (Blondin et al., 1996). Here, oocytes from follicles following 20 hours of coasting displayed a lower blastocyst rate than those from the plateau (68 hours). Accordingly, oocytes obtained at 20 hours of coasting are likely to remain under FSH influence and may not have completed competence differentiation. From 20 to 92 hours, there was no new follicle recruitment. This can logically be explained by the inhibition of larger follicles and the basal LH level, illustrating the functional repression of a dominant cohort (Ginther et al., 2003). Here we observed that the number of follicles was constant throughout coasting although there were more large follicles at 92 hours, consistent with the results of Goodhand et al. (1999). Therefore, we estimate that our coasting period mimics single follicle dominance and prevents the recruitment of new follicles. It is important to remember that FSH reduction during 4 to 5 days is physiological in normal cycling cows (Cooke et al., 1997) and women (Dighe et al., 2005; Stricker et al., 2006). During coasting, all follicles larger than 7-8 mm have a pseudo-dominant status, with LH receptors (Ginther, 2000), and have undergone differentiation under basal LH as in normal cycles. However, since our protocols are initiated when progesterone is high, the amplitude of LH pulses does not increase, and consequently these follicles will eventually regress.

Using in vitro maturation of immature bovine oocytes, the average reported blastocyst rate has not often exceeded 30% over the last 25 years (Sirard at al., 1988; Rizos et al., 2002). Here we define coasting limitations while at the same time highlighting coasting benefits. Our results indicate an oocyte competence window between 44 and 68 hours of coasting with the best results for a coasting period of 54+/−7 hours. According to our mathematical model, an optimal statistical blastocyst rate (70%) can be reached with these conditions. Considering blastocyst rate and data dispersion on one hand and coasting timing as a categorical mathematical variable on the other, the best period revealed here was 44 hours with a mean outcome of 71% blastocysts, which is relevant in the superstimulation IVM-IVF context. This rate is higher in comparison to in vivo produced and fertilized oocytes in an ovary stimulation context (60%) (Sirard et al., 1985) and is close to the success rate of natural-cycle-produced oocytes (80%) (Merton et al., 2003).

In this experiment, half of the oocytes, randomly chosen, were frozen for subsequent RNA analysis (see Example 2) which limits the absolute number of embryos produced in this study. The theoretical blastocyst outcome (that would apply the same development rate to all oocytes obtained)—i.e., the embryo amount per ovum pick up (OPU)—is 13.3 at 44 hours of coasting which is higher than the best estimation for OPU-MOET, 5-8 (Merton et al., 2003). This difference could be partly explained by the fact that in our study, the cows used were relatively young and fertile. These results also indicate that, if not in the optimal period, coasting may lead to reduced developmental capacity. These observations call for further analysis to identify the related causes of such oocyte changes.

In summary, our results provide new information about the importance of FSH decrease in oocyte competence acquisition. First, we showed that the precise coasting duration defined here was a key element to obtain the best success rates. Secondly, decreased oocyte quality occurred in a continuous follicular growth context, reinforcing previous observations showing the negative impact of artificially-extended follicle life. Therefore, we demonstrated here that the optimal period between FSH surge and transvaginal aspiration is 54+/−7 hours and that a well-defined competence window is crucial to obtain optimal oocyte quality in ovarian stimulated milking cows.

### Example 2

### Markers in Bovine Follicular Cells, Cumulus Cells Associated with Mature Oocytes

### Materials and Methods

### RNA Extraction and Amplification

From the six animals used in the study described in Example 1, three were chosen for microarray analysis, mainly based on the amount of oocytes available in each treatment. Pools of four to ten oocytes from three out of the six animals were used in the microarray analysis (and the three other animals will be used for real-time PCR validation). Total RNA was extracted with Pico-Pure™ RNA Isolation Kit (Applied Biosystems, Carlsbad, Calif., USA) following the manufacturer's protocol and including DNase treatment on the purification column. Total RNA integrity and concentration were evaluated on a 2100-Bioanalyzer™ (Agilent Technologies, Palo Alto, Calif., USA) with the RNA PicoLab Chip™ (Agilent Technologies). To generate enough material for hybridisation, the samples were amplified. Antisense RNA was produced using the RiboAmp HS™ RNA amplification kit (Applied Biosystems). After two amplification round of 6 h each, the aRNA output was quantified using the NanoDrop ND1000™ (NanoDrop Technologies, Wilmington, Del., USA).

### Sample Labeling and Microarray Hybridization

For each sample, 2 μg of aRNA were labelled using the ULS™ Fluorescent Labelling Kit for Agilent arrays (with Cy3 and Cy5) (Kreatech Diagnostics, Amsterdam, Netherlands). The labelled product was then purified with the Pico-Pure™ RNA Isolation Kit but without DNase treatment. Labelling efficiency was measured using the Nano-Drop ND-1000™. Samples from the 3 biological replicates were hybridized on EmbryoGENE™'s bovine 44K microarray (Robert et al. 2011, Mol. Reprod. Dev. (in press)). Each hybridization was performed in the following design: for each cow individually, each coasting time was compared to others (i.e. 20 hrs vs 44 hrs; 20 hrs vs 68 hrs; 20 hrs vs 92 hrs; 44 hrs vs 68 hrs; 44 hrs vs 92 hrs and 68 hrs vs 92 hrs) for a total of six comparisons (FIG. 1, Hybridization design). Overall, 36 hybridizations, corresponding to the three cows and six comparisons were done using a dye-swap set-up. A total of 825 ng of each labelled sample (Cy3 and Cy5) were incubated in a solution containing 2× blocking agent and 5× fragmentation buffer in a volume of 55 μl at 60° C. for 15 minutes and were put on ice immediately after. 55 μl of 2× GEx Hybridization Buffer HI-RPM was added for a total volume of 110 μl. The hybridization mix (100 μl) was added onto the array and hybridization was performed at 65° C. for 17 h using an Agilent™ Hybridization chamber in a rotating oven. Slides were then washed with Gene Expression™ Wash Buffer 1 containing 0.005% Triton X-102™ for three minutes at room temperature and then transferred to Gene Expression™ Wash Buffer 2 containing 0.005% Triton X-102™ for three minutes at 42° C. Final washes with acetonitrile for 10 sec at room temperature and with drying and stabilization solution for 30 sec at room temperature were performed before air-drying of the slides. The slides were scanned using the Tecan PowerScanner™ microarray scanner (Tecan Group ltd, Mannerdorf, Switzerland) and features were extracted using ArrayPro 6.4™ (Media Cybernetics, Bethesda, Md., USA).

### Microarray Data Analysis

Microarray data were submitted to a simple background substraction, a Loess within array normalization, statistically analyzed using Limma package in reference design (20 hrs). Foreground mean intensities and median background intensities were used (default settings in Limma), the average of technical replicates were used. F value (ANOVA) was calculated.

Positive probe signals were determined with normalized data using the cut-off log 2 of the expression of 7. All the probes equal or lower than the threshold cut-off were considered to be linked to absent transcript.

cDNA Preparation and Quantitative RT-PCR

Total RNA from cumulus and granulosa cells and from pools of two to ten oocytes was extracted with Pico-Pure™ RNA Isolation Kit and directly reverse transcribed using q-Script Flex™ cDNA Synthesis Kit (Quanta Biosciences, Gaithersburg, Md., USA) with oligo dT₍₂₀₎ primers following manufacturer's recommendations. One ng of total RNA was used for the reverse transcription for the cumulus and granulosa cells. At the end of the cumulus RNA reverse transcription, 40 μl of nuclease-free water were added to the final 20 μl RT reaction.

50 ng of aRNA was reverse transcribed using q-Script Flex™ cDNA Synthesis Kit (Quanta Biosciences, Gaithersburg, Md., USA) with random primers following manufacturer's recommendations. The primers used for real-time RT-PCR were designed using the IDT PrimerQuest™ tool (available at Integrated DNA technologies website) from sequences obtained using the UMD3.1/bosTau5 assemble version of the bovine genome and results from our microarray analysis. To confirm the specificity of each pairs of primers, electrophoresis on a standard 1.2% agarose gel was performed for each amplified fragment. The PCR product was then purified with the QIAquick™ Gel Extraction kit (Qiagen), quantified using the NanoDrop ND1000™ and sequenced. The products were used to create the standard curve for quantification experiment, with dilutions ranging from 2×10⁻⁴ to 2×10⁻⁸ ng μl⁻¹. Real-time PCR was performed on a LightCycler 480™ (Roche Diagnostics, Laval, QC, Canada) using SYBR incorporation. Each reaction, in a final volume of 20 contained the cDNA corresponding to 0.025 oocyte or 2 μl of cDNA for granulosa cells, 0.25 mM of each primer and 1×SYBR mix (LightCycler 480™ SYBR Green I Master™, Roche Diagnostics). The PCR conditions used for all genes were as follows: denaturing cycle for 10 min at 95° C.; 50 PCR cycles (denaturing, 95° C. for 1 s; annealing for 5 s; extension, 72° C. for 5 s), a melting curve (94° C. for 5 s, 72° C. for 30 s and a step cycle starting at 72° C. up to 94° C. at 0.2° C./s) and a final cooling step at 40° C. Complementary DNA quantification was performed with the LightCycler 480™ Software Version 1.5 (Roche Diagnostics) by comparison with the standard curve. For cumulus cells, real-time PCR was performed on a LightCycler 2.0™ (Roche Diagnostics, Laval, QC, Canada) using SYBR incorporation. Each reaction, in a final volume of 20 μl, contained the 2 μl of cDNA, 0.125 mM of each primer and 2 μl of SYBR Green mix, 3 mM of MgCl2 (LightCycler™ FastStartDNA Master SYBR Green I Kit™, Roche Diagnostics). The PCR conditions used for all genes were as follows: denaturing cycle for 10 min at 95° C.; 50 PCR cycles (denaturing, 95° C. for 5 s; annealing for 5 s; elongation, 72° C. for 30 s, acquisition for 3 s), a melting curve (95° C. for 1 s, 65° C. for 5 s and a step cycle starting at 65° C. up to 95° C. at 0.1° C./s) and a final cooling step at 40° C.) and a final cooling step at 40° C. Complementary DNA quantification was performed with the LightCycler® Software Version 4.1 (Roche Diagnostics) by comparison with the standard curve. PCR specificity was confirmed by melting-curve analysis.

### Statistical Analysis of RT-PCR Results

For each gene tested, three biological replicates were used. Analysis of gene expression stability over oocytes from the three different cows was performed using the GeNorm VBA™ applet software as described by Vandesompele et al. (2002). The most stable reference genes were identified by the stepwise exclusion of the least stable gene and recalculating the M values. Following GeNorm™ analysis, ACTB, GAPDH and H2A.Z were the most stable genes for the oocyte with M values <1.6 as recommended by the software (M value=0.650). For the granulosa cells, ACTB and GAPDH were the most stable genes with M value=1.6. For cumulus cells ACTB, GAPDH and/or PPHLN1 (depending of the way to analyze data, e.g chronological progression or minimum vs. maximum blastocyst rate) were the most stable genes with M value <1.1. Evaluation of mRNA differences between the four coasting times was performed by an ANOVA followed by a Tukey post-hoc test with Graph Pad Prism™ Version 5.0. Evaluation of mRNA differences between the minimum and maximum blastocyst rate was performed by a t-test. Differences were considered to be statistically significant at the 95% confidence level (p<0.05). Data are presented as mean±s.e.m.

### Results

Positive probe signals were determined on normalized data by establishing a significant threshold of cut-off based on a degree of confidence associated with the variability of the negative controls. This cut-off threshold was calculated as follows: T=M+2× S.D., where T is the calculated threshold for cut-off, M is the average of the intensities of negative controls present on the slides and S.D. is the Standard Deviation. All the data equal or lower to the cut-off threshold.

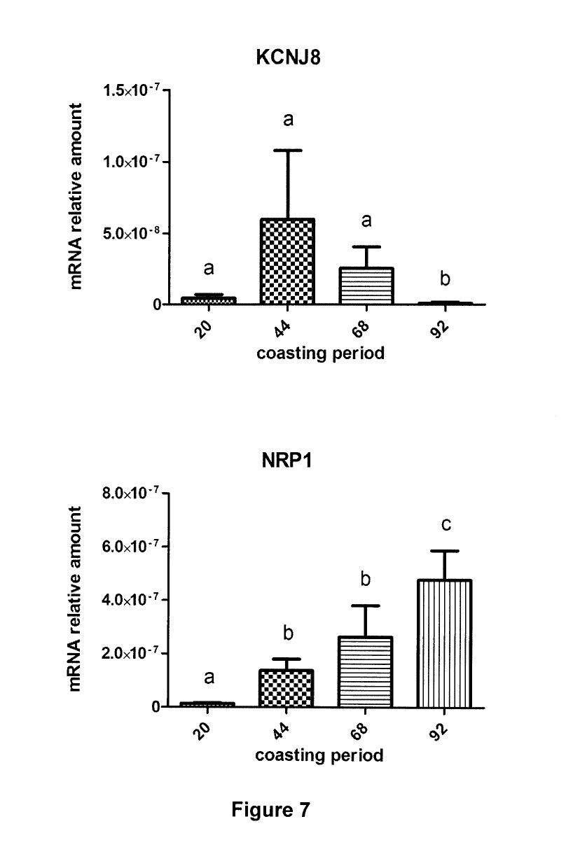

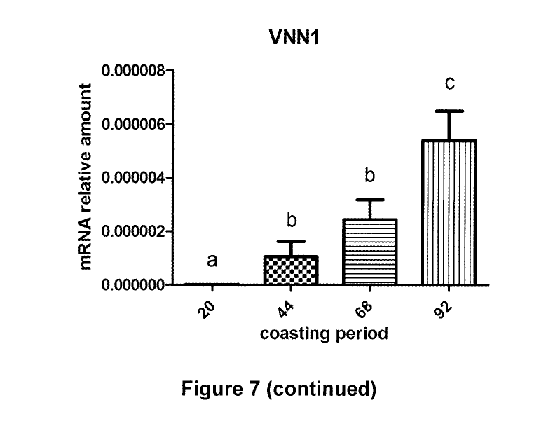

Tables I, II, and III describe the genes that were found to be of interest for diagnostic purpose in the 3 different tissues, namely granulosa cells, cumulus cells and oocytes. These list contains only genes that were found to be significantly regulated in relation to follicular maturity using the different times in a classic anova analysis. As shown in FIG. 7, the genes KCNJ8, NRP1, VNN1 were validated in RT-PCR.

TABLE I
Follicular Cells markers - Granulosa cells

NCBI GeneID

| Gene | Bos | Homo |
|---|---|---|
| symbol | Taurus | sapiens |
| A2M | 513856 | 2 |
| ADAMTS1 | 512171 | 9510 |
| ADAMTSL5 | 616145 | 339366 |
| ADCY7 | 281603 | 600385 |
| ANGPT2 | 282141 | 285 |
| ANK3 | 511203 | 288 |
| ANKH | 511800 | 56172 |
| ANKRD1 | 510376 | 27063 |
| ANXA1 | 327662 | 301 |
| ANXA3 | 518050 | 106490 |
| ANXA8 | 281627 | 653145 |
| APOD | 613972 | 347 |
| ARRB1 | 281637 | 408 |
| AXL | 516598 | 558 |
| BCL2A1 | 282151 | 597 |
| BMP2 | 615037 | 112261 |
| BMP6 | 617566 | 654 |
| BMPR1B | 407128 | 658 |
| BTG2 | 539364 | 7832 |
| CARD6 | 520291 | 84674 |
| CASP4 | 338039 | 837 |
| CASP8 | 507481 | 841 |
| CD1E | 510832 | 913 |
| CD47 | 282661 | 961 |
| CD53 | 505040 | 963 |
| CD99 | 509230 | 4267 |
| CHODL | 613942 | 140578 |
| CIRBP | 507120 | 1153 |
| CLDN11 | 508268 | 5010 |
| CNIH3 | 615236 | 149111 |
| COBLL1 | 532067 | 22837 |
| CTGF | 281103 | 1490 |
| CXCR4 | 281736 | 7852 |
| DAB2 | 509221 | 1601 |
| DAP | 616066 | 1611 |
| DCLK1 | 613449 | 9201 |
| DCN | 280760 | 1634 |
| DUSP1 | 539175 | 1843 |
| EFEMP1 | 511566 | 2202 |
| EGR1 | 407125 | 1958 |
| EMCN | 616367 | 51705 |
| FABP4 | 281759 | 2167 |
| FABP5 | 281760 | 2171 |
| FN1 | 280794 | 2335 |
| FOXO1 | 506618 | 2308 |
| GFPT2 | 530101 | 9945 |
| GLIPR1 | 767905 | 11010 |
| HPCAL1 | 513870 | 3241 |
| IGF2 | 281240 | 3481 |
| IGJ | 280821 | 3512 |
| IL1A | 281250 | 3552 |
| IL1RN | 281860 | 3557 |
| ISG15 | 281871 | 9636 |
| JAG1 | 783681 | 182 |
| KCNJ8 | 282572 | 3764 |
| LRP8 | 407179 | 7804 |
| LRRC17 | 777690 | 10234 |
| LUM | 280847 | 4060 |
| MAML2 | 521194 | 84441 |
| MMP25 | 531092 | 64386 |
| MXRA7 | 617087 | 439921 |
| MXRA8 | 522392 | 54587 |
| NID2 | 521854 | 22795 |
| NOV | 505727 | 4856 |
| NRP1 | 539369 | 8829 |
| NTS | 280881 | 4922 |
| NUCB1 | 505351 | 4924 |
| OGN | 280884 | 4969 |
| OLFM1 | 507002 | 10439 |
| OLR1 | 281368 | 4973 |
| OSTF1 | 281961 | 26578 |
| PDCD4 | 506724 | 27250 |
| PDE8B | 1E+08 | 8622 |
| PDK4 | 507367 | 5166 |
| PICALM | 513579 | 8301 |
| PLAT | 281407 | 173370 |
| PPL | 522886 | 5493 |
| PRG4 | 280867 | 10216 |
| PROCR | 282005 | 10544 |
| PRSS23 | 538575 | 11098 |
| PTGER4 | 282331 | 5734 |
| RARRES1 | 510102 | 5918 |
| RELN | 281450 | 5649 |
| RGS1 | 540836 | 5996 |
| SCIN | 281478 | 85477 |
| SELP | 281486 | 6403 |
| SEPP1 | 282066 | 6414 |
| SERINC3 | 511861 | 10955 |
| SFRP1 | 282068 | 6422 |
| SGK1 | 515854 | 6446 |
| SIPA1L2 | 535132 | 57568 |
| SLC40A1 | 527023 | 30061 |
| SMAGP | 787004 | 57228 |
| SNAP23 | 522423 | 8773 |
| SNCAIP | 540156 | 9627 |
| SORL1 | 533166 | 6653 |
| SPARC | 282077 | 6678 |
| SPP1 | 281499 | 6696 |
| STAR | 281507 | 6770 |
| SVIL | 281509 | 6840 |
| TANK | 539513 | 10010 |
| TF | 280705 | 7018 |
| TFPI2 | 360007 | 7980 |
| TGFB3 | 538957 | 7043 |
| TGFBR2 | 535376 | 7048 |
| TGFBR3 | 784894 | 7049 |
| THBD | 281529 | 7056 |
| TLR2 | 281534 | 7097 |
| TNFRSF21 | 537922 | 27242 |
| TNFSF9 | 521748 | 8744 |
| TRAF6 | 539124 | 7189 |
| TRIB1 | 521857 | 10221 |
| TRIB2 | 352960 | 28951 |
| VAT1 | 510698 | 10493 |
| VNN1 | 526704 | 8876 |

TABLE II
Follicular Cells markers - Cumulus cells

NCBI GeneID

| | Bos | |
|---|---|---|
| Gene symbol | taurus | Homo sapiens |
| MAN1A1 | 530027 | 4121 |
| B2M | 280729 | 567 |
| GATM | 414732 | 2628 |
| RELN | 281450 | 5649 |
| ISG12(A) or IFI27 | 507138 | 3429 |
| NSDHL | 616694 | 18194 |
| ANK3 | 511203 | 288 |
| CYP11A1 | 338048 | 1583 |
| LOC286871 | 286871 | N/A |
| VNN1 | 526704 | 8876 |
| FADS2 | 521822 | 9415 |
| CNN1 | 534583 | 1264 |
| PCYT2 | 510274 | 5833 |
| RETSAT | 614455 | 54884 |
| CLIC3 | 505436 | 9022 |
| TGFBR3 | 784894 | 7049 |
| PARP12 | 513185 | 64761 |
| NRP1 | 539369 | 8829 |
| INSIG1 | 511899 | 3638 |
| HSD17B1 | 353107 | 3292 |
| APOA1 | 281631 | 335 |
| HN1 | 613381 | 15374 |
| PFKP | 507119 | 5214 |
| CRAT | 512902 | 1384 |
| LONP1 | 510796 | 9361 |
| MRP63 | 615666 | 78988 |
| PPHLN1 (House Keeping) | 532695 | 51535 |
| ACTB (House Keeping) | 280979 | 60 |
| GAPDH (House Keeping) | 281181 | 2597 |

TABLE III
Oocyte markers

| | |
|---|---|
| Gene | NCBI GeneID |

| symbol | Bos Taurus | Homo sapiens |
|---|---|---|
| ENY2 | 614069 | 56943 |
| MAD2L2 | 506605 | 10459 |
| CDK1 | 281061 | 983 |
| NLRP5 | 493717 | 126206 |
| AURKAIP1 | 506359 | 54998 |
| HAUS8 | 511056 | 93323 |
| TCEA1 | 505722 | 6917 |
| TFE3 | 520800 | 7030 |
| NFYB | 614382 | 4801 |
| PDE6C | 281975 | 5146 |
| SKP1 | 615427 | 6500 |
| MAD2 | 780876 | 4085 |
| FZR1 | 530333 | 51343 |
| CDC26 | 777693 | 246184 |
| ARRB2 | 281638 | 409 |
| MATR3 | 505129 | 9782 |
| TAF1A | 505051 | 9015 |
| TFDP1 | 534579 | 7027 |
| THOC6 | 100125940 | 79228 |
| PHC2 | 537511 | 1912 |
| LSM10 | 618089 | 84967 |
| EPC2 | 539432 | 26122 |

### Example 3

### Use of a Chip Comprising Antibodies for Assessing Maturity of Ovarian Follicles

This hypothetical example describes the use of a solid support such as a chip for evaluating the competence of a mammalian oocyte.

A chip (e.g. Ciphergen ProteinChip™) for measuring two or more predetermined ovarian markers is prepared using known methods (e.g. Lin et al., Application of SELDI-TOF mass spectrometry for the identification of differentially expressed proteins in transformed follicular lymphoma. Mod Pathol. 2004 June; 17(6):670-8; Wang et al., Mass spectrometric analysis of protein markers for ovarian cancer. Clin Chem. 2004 October; 50(10):1939-42; Simonsen et al., Amyloid beta 1-40 quantification in CSF: comparison between chromatographic and immunochemical methods. Dement Geriatr Cogn Disord. 2007; 23(4):246-50)

The chip comprises a plurality of antibodies types, each type being capable of specifically binding to a predetermined ovarian marker (e.g. specific for polypeptides expressed by the gene of interest). The chip is contacted with a cell lysate or with biological fluids from cumulus cells, follicular cells (e.g. follicular fluid) and/or oocyte(s). After a certain period the chip is rinsed for removing unbound non-specific material and it is submitted to mass spectrometry for quantification of the materials remaining on the chip. Results from the quantification measurements are inputted into a computer for analysis using a multivariable algorithm for obtaining a score. The score gives an indication of the maturity of the mammalian follicle.

### Example 4

### Use of a DNA Chip for Evaluating Maturity of Ovarian Follicles

This hypothetical example describes the use of a solid support such as a DNA chip for evaluating the competence of a mammalian oocyte.

A DNA chip (e.g. micro-array with cDNA or oligomers) for measuring two or more predetermined ovarian markers is prepared using known methods (e.g. Harry et al., Predicting the response of advanced cervical and ovarian tumors to therapy. Obstet Gynecol Surv. 2009 August; 64(8):548-60; Ross J S. Multigene classifiers, prognostic factors, and predictors of breast cancer clinical outcome. Adv Anat Pathol. 2009 July; 16(4):204-15; Sotiriou C and Pusztai L. Gene-expression signatures in breast cancer. N Engl J. Med. 2009 Feb. 19; 360(8):790-800).

The chip comprises a plurality of specific DNA targets (each target being capable of specifically binding to a predetermined ovarian marker (e.g. a cDNA molecule or a mRNA molecule hybridizing specifically with a mRNA expressed by the gene of interest). The chip is contacted with a set of DNA targets (e.g. cDNA or mRNA molecules having about 20, 30, 40, 50, 60, 70 or more nucleotides) and probed with complementary DNA obtained by reverse transcription/amplification of the RNA expressed in the selected tissues (oocyte, follicular or cumulus cells) to examine fluorescent dyes intensity. After a certain period the chip is rinsed for removing unbound non-specific material and it is submitted to laser in a slide reader for pixel quantification of the materials remaining on the chip. Results from the quantification measurements are inputted into a computer for analysis using a multivariable algorithm for obtaining a score. The score gives an indication of the maturity of the mammalian follicle.

### Example 5

### Optimization of Controlled Ovarian Stimulation (COS) and In Vitro Maturation (IVM)

This hypothetical example describes the use of the ovarian markers of the invention for evaluating follicular maturity status after a first COS and optimize a subsequent COS in a human patient in the course of assisted reproduction.

In a human fertility clinical environment, selected markers are used on a pool of follicular cells from a patient to assess the follicular maturity status after an initial or ensuing ovarian stimulation to recover oocytes. The sample is analysed locally for assessing one or more marker or send to a central lab for more a complex analysis. Since the result of the test is not required immediately, the physician can decide to wait for the embryo transfer (resulting in pregnancy or not) before sending the sample for analysis. The results of the marker(s) assessment and follicular maturity status are be used to better prepare the medication and protocol for the subsequent COS if required (i.e. no pregnancy).

In a human fertility performing IVM, the marker(s) analysis is used to assess the follicular maturity of a pool of follicles even if the LH surge has not been triggered. A rapid test is used so the physician can decide the oocyte IVM period or the type of in vitro culture conditions that would benefit the cultured oocytes. Such analysis is done on individual follicles or cumulus cells to individually classify oocytes upon their follicular maturity.

### Example 6

### Markers in Human Follicular Cells, from Small Medium or Large Follicles Associated with Transferable or not Transferable Embryos

### Materials and Methods

All chemicals were obtained from Sigma-Aldrich (St. Louis, Mo.), unless otherwise stated

### Samples

Results presented here are based on human samples. Cells were recovered (Centre de Fertilité d'Ottawa, Dr Marie-Claude Leveillé) on a per follicle basis by individual follicle puncture, and individual data collection such as follicular fluid volume,

In Vitro Fertilization outcome. Follicles were sorted with 2 criteria, size group and related embryo outcome, see details in Table VI.

TABLE VI

| | Small | Medium | Large | Medium |
|---|---|---|---|---|
| | follicles | follicles | follicles | follicles |
| Follicle volumes | 0.5-1.5 mL | 2-3 mL | 3.5-5 mL | 2 mL |
| (obtained with OPU) | | | | |
| Equivalent follicle diameters based | 9.8-14.2 mm | 15.6-17.9 mm | 18.8-21.2 mm | 15.6 mm |
| on the follicle volume | | | | |
| Developmental competence | 1PN-10 cells | transferable | 2PN-6 | 1PN |
| associated to the enclosed oocytes | | | cells | |

RNAs of individual follicles were extracted, analyzed, amplified and hybridized on the Whole human genome microarray 4*44 k V2 arrays and the same follicles were used for real-time PCR validation.

### RNA Extraction and Amplification

Total RNA was extracted with miRNeasy™ mini kit following the manufacturer's protocol using miRNeasy™ mini kit (Qiagen, Mississauga, Canada) following the manufacturer's protocol and DNAse digestion was performed using the RNase-Free DNase Set, directly on the extraction column (Qiagen). Total RNA integrity and concentration were evaluated on a 2100-Bioanalyzer™ (Agilent Technologies, Palo Alto, Calif., USA) with the RNA PicoLab Chip™ (Agilent Technologies). To generate enough material for hybridisation, the samples were amplified. Antisense RNA was produced using the RiboAmp HS™ RNA amplification kit (Applied Biosystems). After two amplification rounds of 6 h each, the aRNA output was quantified using the NanoDrop ND-1000™ (NanoDrop Technologies, Wilmington, Del., USA).

### Sample Labeling and Microarray Hybridization

For each sample, 2 μg of aRNA were labelled using the ULS™ Fluorescent Labelling Kit for Agilent arrays (with Cy3 and Cy5) (Kreatech Diagnostics, Amsterdam, Netherlands). The labelled product was then purified with the Pico-Pure™ RNA Isolation Kit but without DNase treatment. Labelling efficiency was measured using the Nano-Drop ND-1000. Samples from the 3 biological replicates were hybridized on the Whole human genome microarray 4*44 k V2 arrays.

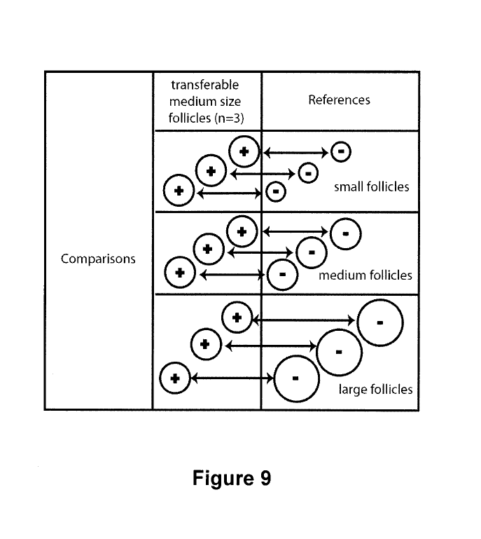

Three comparisons were performed and the experimental design is summarized in FIG. 9: 1) 3 medium size follicle (2 to 3 ml) linked to transferable embryo were compared individually to 3 individual small follicle (0.5 to 1.5 ml) associated with development up to 10 cells; 2) 3 medium size follicle (2 to 3 ml) linked to transferable embryo were compared individually to 3 individual medium size follicle associated with development up to 1 PN 3) 3 medium size follicle linked to transferable embryo were compared individually to 3 individual big follicles (3.5 to 5 ml) associated with development up to 6 cells. Overall, 18 hybridizations, corresponding to the 4 groups of 3 follicles and the 3 comparisons, were done, using a dye-swap set-up. A total of 825 ng of each labelled sample (Cy3 and Cy5) were incubated in a solution containing 2× blocking agent and 5× fragmentation buffer in a volume of 55 μl at 60° C. for 15 minutes and were put on ice immediately after. 55 μl of 2× GEx™ Hybridization Buffer HI-RPM was added for a total volume of 110 μl. The hybridization mix (100 μl) was added onto the array and hybridization was performed at 65° C. for 17 h using an Agilent™ Hybridization chamber in a rotating oven. Slides were then washed with Gene Expression™ Wash Buffer 1 containing 0.005% Triton X-102™ for three minutes at room temperature and then transferred to Gene Expression™ Wash Buffer 2 containing 0.005% Triton X-102™ for three minutes at 42° C. Final washes with acetonitrile for 10 sec at room temperature and with drying and stabilization solution for 30 sec at room temperature were performed before air-drying of the slides. The slides were scanned using the Tecan PowerScanner™ microarray scanner (Tecan Group ltd, Mannerdorf, Switzerland) and features were extracted using ArrayPro™ 6.4 (Media Cybernetics, Bethesda, Md., USA).

### Microarray Data Analysis

Microarray data were submitted to a simple background substraction, a Loess within array normalization, quantile between array normalization, and Limma simple statistical analysis between each comparison. Differences between treatments were considered significant when the Limma P-value was inferior to 0.05.

cDNA Preparation and Quantitative RT-PCR

10 ng of total RNA of each individual follicles used for microarray (excepting one because of limiting RNA amount) was reverse transcribed using q-Script Flex™ cDNA Synthesis Kit (Quanta Biosciences, Gaithersburg, Md., USA) with oligo dT₍₂₀₎ primers following manufacturer's recommendations.)

The primers used for real-time RT-PCR were designed using the IDT PrimerQuest™ tool (available at Integrated DNA Technologies' web site). To confirm the specificity of each pairs of primers, electrophoresis on a standard 1.2% agarose gel was performed for each amplified fragment. The PCR product was then purified with the QIAquick™ Gel Extraction kit (Qiagen), quantified using the NanoDrop ND-1000™ and sequenced. The products were used to create the standard curve for quantification experiment, with dilutions ranging from 2×10⁻⁴ to 2×10⁻⁸ ng Real-time PCR was performed on a LightCycler 480™ (Roche Diagnostics, Laval, QC, Canada) using SYBR incorporation. Each reaction, in a final volume of 20 μl, contained 2 μl (0.2 ng) of the cDNA product, 0.25 mM of each primer and 1× SYBR mix (LightCycler 480™ SYBR Green I Master™, Roche Diagnostics). The PCR conditions used for all genes were as follows: denaturing cycle for 10 min at 95° C.; 50 PCR cycles (denaturing, 95° C. for 1 s; annealing, for 5 s; extension, 72° C. for 5 s), a melting curve (94° C. for 5 s, 72° C. for 30 s and a step cycle starting at 72° C. up to 94° C. at 0.2° C./s) and a final cooling step at 40° C. Complementary DNA quantification was performed with the LightCycler 480™ Software Version 1.5 (Roche Diagnostics) by comparison with the standard curve. PCR specificity was confirmed by melting-curve analysis.

### Statistical Analysis of RT-PCR Results

For each gene tested, three biological replicates and three technical replicates were used. Analysis of gene expression stability over the different follicles was performed using the GeNorm™ VBA applet software as described by Vandesompele et al. (2002). The most stable reference genes were identified by the stepwise exclusion of the least stable gene and recalculating the M values. Following GeNorm™ analysis, ACTB and GAPDH were the most stable genes with M values <1.5 as recommended by the software (M value=0.545).

### Results

### A Posteriori Data Analysis

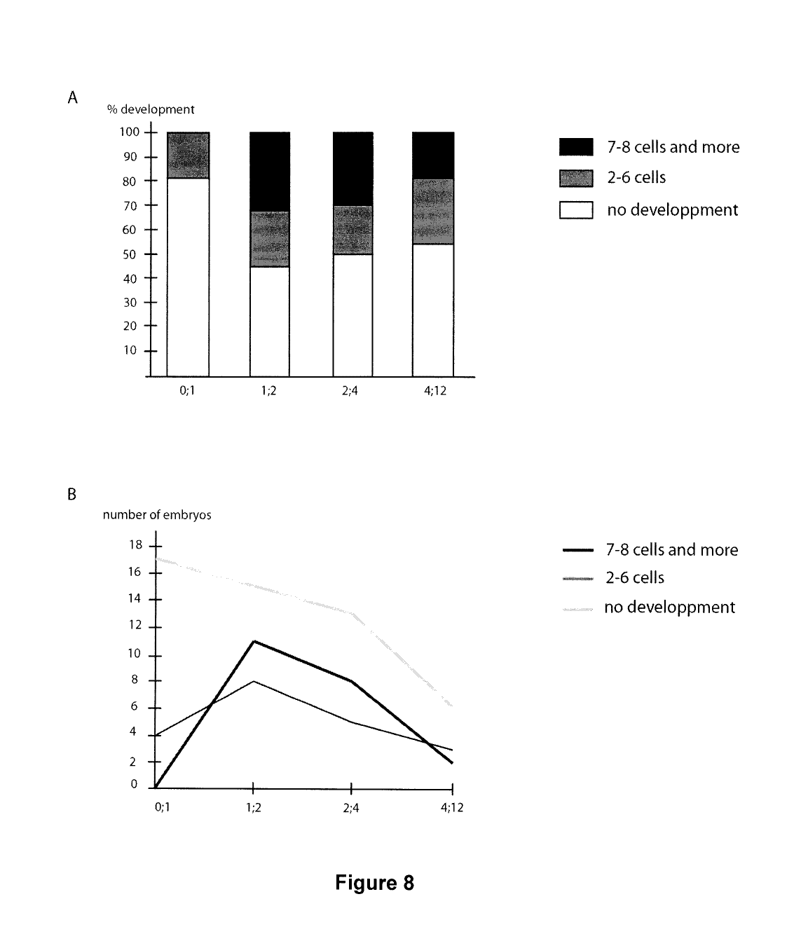

Follicles were classified in 4 follicle size groups and 3 developmental competence groups, and data were organized in relative proportion FIG. 8A, in absolute FIG. 8B, and synthesized in a table (Table VII).

For the smallest follicles, follicle proportion associated to no embryo development is maximal comparing to the other size group. In parallel, the follicle proportion associated the highest development is the smallest comparing to the other embryo development categories. This follicle size category 0; 1 can be considered the less competent one.

For the biggest follicles, the proportion of follicles associated to the highest development (7-8 cells and more) is the second smallest comparing to the other follicle size group. In parallel, follicle proportion associated to no embryo development is the second biggest comparing to the other size group. This follicle size category 4; 12 is the second less competent one.

The 2 medium size follicle groups, 1-2 ml and 2-4 ml are the only ones associated to more 7-8 cells and more embryo development than 2-6 cells embryos. Furthermore, the proportion of 7-8 cells and more is approximately 30% in these medium size follicles, comparing to 20% for the biggest follicles, 4-12 ml. According to our data, the medium size follicles are effectively associated to the best oocyte developmental competence

TABLE VII
Absolute and relative data presented in FIGS. 8A and 8B

| | Estimated diameter (mm) | | | | | | | | |
|---|---|---|---|---|---|---|---|---|---|
| | 0; 12.4 | | 12.4; 15.6 | | 15.6; 17.9 | | 19.7; 21.2 | | |
| | Follicular fluid volume (mL) | | | | | | | | |
| | 0; 1 | | 1; 2 | | 2; 4 | | 4; 12 | | total samples |
| No | 17/21 | (80.9%) | 15/34 | (44.1%) | 13/26 | (50%) | 6/11 | (54.5%) | 37 |
| development | | | | | | | | | |
| 2-6 cells | 4/21 | (19%) | 8/34 | (23.5%) | 5/26 | (19.2%) | 3/11 | (27.3%) | 35 |
| 7-8 cells | 0/21 | (0%) | 11/34 | (32.3%) | 8/26 | (30.8%) | 2/11 | (18.2%) | 20 |
| and more | | | | | | | | | |
| sample size | 21 | (100%) | 34 | (100%) | 26 | (100%) | 11 | (100%) | 92 |

### Microarray Analysis

Results of the microarray analysis are summarized in Table VIII.

TABLE VIII

| | | | | | Differential expression | |
|---|---|---|---|---|---|---|
| | Upregulation | | Downregulation | | | Relative to total |
| | abso- | rela- | abso- | rela- | Abso- | (n = 21851) |
| Size* | lute | tive | lute | tive | lute | gene symbol |
| M+/P− | 65 | 42.8% | 87 | 57.2% | 152 | 0.7% |
| M+/G− | 108 | 65.1% | 58 | 34.9% | 166 | 0.8% |
| M+/M− | 595 | 37.9% | 973 | 62.0% | 1568 | 7.2% |

*Size: m+: medium size follicles associated to transferable embryos, m−: medium size follicles not associated to transferable embryos p−: small follicles not associated to transferable embryos, g−: large follicles not associated to transferable embryos.

For the inter size comparisons (M+/P− and M+/G−) there was approximately 150 differentially expressed genes, representing less than 1% of the probes, and including respectively 2/5 and 2/3 of upregulated genes (p-value<0.05 and fold change>1.5), Table VIII.

For the intra-size comparison (M+/M−), 7.2% of the probes are differentially expressed, including 2/5 of upregulated genes (p-value<0.05 and fold change >1.5), Table VIII.

Crossing the results, we identified 2 genes upregulated in each comparison in the medium size follicles associated to transferable embryos, PDE8B and ZNF880. THBD was among the genes differentially expressed in M+/M− and in M+/P− comparisons. Candidate for Q-PCR validation were chosen on cross-comparisons data from this study and a previous study in cow (Example 2). PDE8B was added because relevant in human but not investigated in bovine for practical reasons (no PDE8B probe on the EmbryoGENE™'s bovine 44K microarray).

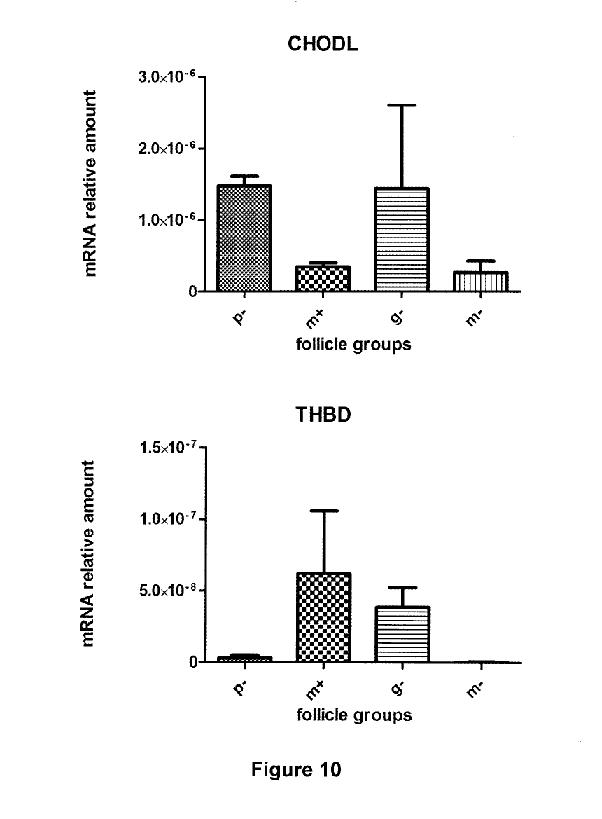

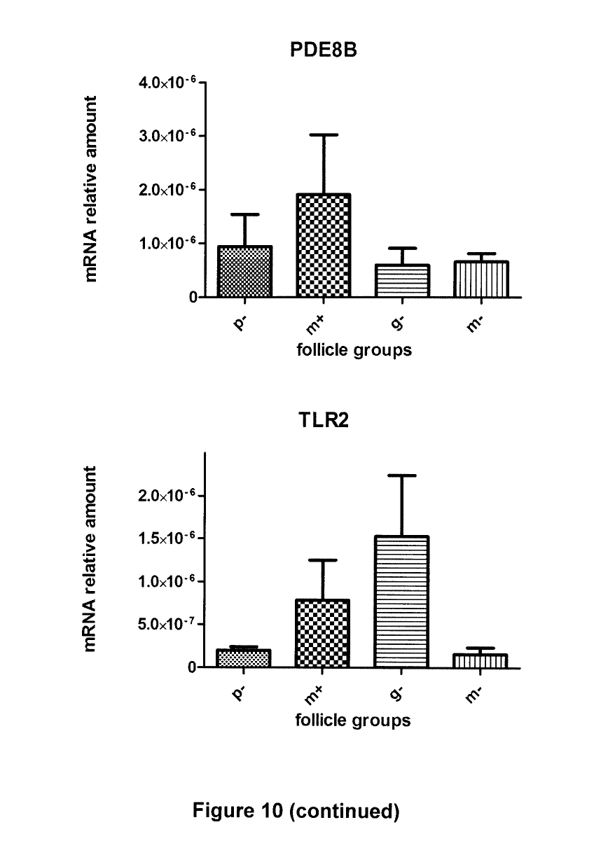

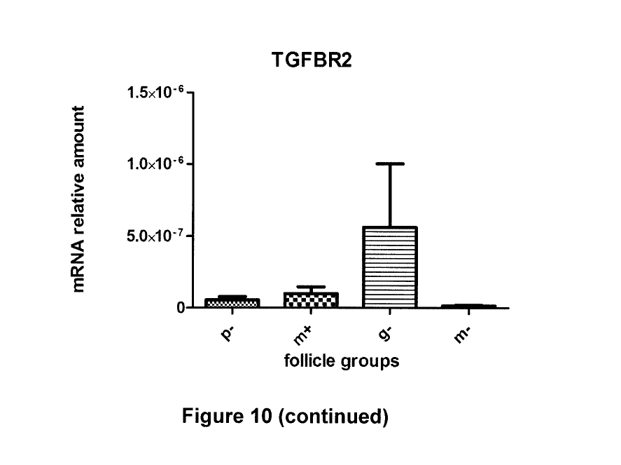

The following genes were found to be of particular interest for diagnostic purpose in granulosa cells: PDE8B, THBD, TLR2, CHODL and TGFBR2. This list contains only genes that were found to be significantly regulated in relation to follicular size and related embryo transferability. This gene transcript study permits to: 1) identify medium size follicles associated to transferable embryos from small and large follicles not associated to transferable embryos; and 2) to distinguish between medium size follicle associated to transferable embryo and medium size follicle not associated to transferable embryo. QPCR results are presented FIG. 10.

### REFERENCES

Blondin P, Bousquet D, Twagiramungu H, Barnes F & Sirard M A 2002 Manipulation of follicular development to produce developmentally competent bovine oocytes. Biol Reprod 66 38-43.
Blondin P, Coenen K, Guilbault L A & Sirard M A 1996 Superovulation can reduce the developmental competence of bovine embryos. Theriogenology 46 1191-1203.
Blondin P, Coenen K, Guilbault L A & Sirard M A 1997 In vitro production of bovine embryos: developmental competence is acquired before maturation. Theriogenology 47 1061-1075.
Blondin P & Sirard M A 1995 Oocyte and follicular morphology as determining characteristics for developmental competence in bovine oocytes. Mol Reprod Dev 41 54-62.
Boichard D B A, Briend M. 2002 Bilan phénotypique de la fertilité chez les bovins laitiers. In Colloque de l'Association pour l'Etude de la Reproduction Animale. Paris, 2002
Bridges P J & Fortune J E 2003 Characteristics of developing prolonged dominant follicles in cattle. Domest Anim Endocrinol 25 199-214.
Cooke D J, Crowe M A & Roche J F 1997 Circulating FSH isoform patterns during recurrent increases in FSH throughout the oestrous cycle of heifers. J Reprod Fertil 110 339-345.
Dighe A S, Moy J M, Hayes F J & Sluss P M 2005 High-resolution reference ranges for estradiol, luteinizing hormone, and follicle-stimulating hormone in men and women using the AxSYM assay system. Clin Biochem 38 175-179.
Ginther O J 2000 Selection of the dominant follicle in cattle and horses. Anim Reprod Sci 60-61 61-79.
Ginther O J, Beg M A, Donadeu F X & Bergfelt D R 2003 Mechanism of follicle deviation in monovular farm species. Anim Reprod Sci 78 239-257.
Goodhand K L, Watt R G, Staines M E, Hutchinson J S & Broadbent P J 1999 In vivo oocyte recovery and in vitro embryo production from bovine donors aspirated at different frequencies or following FSH treatment. Theriogenology 51 951-961.
Hagemann L J, Beaumont S E, Berg M, Donnison M J, Ledgard A, Peterson A J, Schurmann A & Tervit H R 1999 Development during single IVP of bovine oocytes from dissected follicles: interactive effects of estrous cycle stage, follicle size and atresia. Mol Reprod Dev 53 451-458.
Lonergan P, Monaghan P, Rizos D, Boland M P & Gordon I 1994 Effect of follicle size on bovine oocyte quality and developmental competence following maturation, fertilization, and culture in vitro. Mol Reprod Dev 37 48-53.
Merton J S, de Roos A P, Mullaart E, de Ruigh L, Kaal L, Vos P L & Dieleman S J 2003 Factors affecting oocyte quality and quantity in commercial application of embryo technologies in the cattle breeding industry. Theriogenology 59 651-674.
Oussaid B, Lonergan P, Khatir H, Guler A, Monniaux D, Touze J L, Beckers J F, Cognie Y & Mermillod P 2000 Effect of GnRH antagonist-induced prolonged follicular phase on follicular atresia and oocyte developmental competence in vitro in superovulated heifers. J Reprod Fertil 118 137-144.
Pavlok A, Kopecny V, Lucas-Hahn A & Niemann H 1993 Transcriptional activity and nuclear ultrastructure of 8-cell bovine embryos developed by in vitro maturation and fertilization of oocytes from different growth categories of antral follicles. Mol Reprod Dev 35 233-243.
Rizos D, Fair T, Papadopoulos S, Boland M P & Lonergan P 2002 Developmental, qualitative, and ultrastructural differences between ovine and bovine embryos produced in vivo or in vitro. Mol Reprod Dev 62 320-327.
Salha O, Nugent D, Dada T, Kaufmann S, Levett S, Jenner L, Lui S & Sharma V 1998 The relationship between follicular fluid aspirate volume and oocyte maturity in in-vitro fertilization cycles. Hum Reprod 13 1901-1906.
Sirard M-A & Trounson A 2003. Follicular factors affecting oocyte maturation and developmental competence. In Biology and Pathology of the Oocyte: Its Role in Fertility and Reproductive Medecine, pp. 305-315.
Sirard M A, Parrish J J, Ware C B, Leibfried-Rutledge M L & First N L 1988 The culture of bovine oocytes to obtain developmentally competent embryos. Biol Reprod 39 546-552.
Sirard M A, Richard F, Blondin P & Robert C 2006 Contribution of the oocyte to embryo quality. Theriogenology 65 126-136.
Sirois J & Fortune J E 1990 Lengthening the bovine estrous cycle with low levels of exogenous progesterone: a model for studying ovarian follicular dominance. Endocrinology 127 916-925.
Stricker R, Eberhart R, Chevailler M C, Quinn F A & Bischof P 2006 Establishment of detailed reference values for luteinizing hormone, follicle stimulating hormone, estradiol, and progesterone during different phases of the menstrual cycle on the Abbott ARCHITECT analyzer. Clin Chem Lab Med 44 883-887.
van de Leemput E E, Vos P L, Hyttel P, van den Hurk R, Bevers M M, van der Weijden G C & Dieleman S J 2001 Effects of brief postponement of the preovulatory LH surge on ovulation rates and embryo formation in eCG/prostaglandin-treated heifers. Theriogenology 55 573-592.
Walsh S W, Williams E J & Evans A C 2011 A review of the causes of poor fertility in high milk producing dairy cows. Anim Reprod Sci 123 127-138.

Headings are included herein for reference and to aid in locating certain sections These headings are not intended to limit the scope of the concepts described therein under, and these concepts may have applicability in other sections throughout the entire specification Thus, the present invention is not intended to be limited to the embodiments shown herein but is to be accorded the widest scope consistent with the principles and novel features disclosed herein.

Unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contain certain errors resulting from variations in experiments, testing measurements, statistical analyses and such.

It is understood that the examples and embodiments described herein are for illustrative purposes only and that various modifications or changes in light thereof will be suggested to persons skilled in the art and are to be included within the present invention and scope of the appended claims.

1. A method for improving ovarian stimulation in a human subject, the method comprising:
obtaining an oocyte and/or follicular cell(s) from an ovarian follicle subsequent to a first controlled ovarian stimulation (COS);
assessing maturity of said ovarian follicle by determining expression level of at least one ovarian marker indicative of said maturity;
optimizing a second COS based on the assessed maturity of the ovarian follicle obtained following the first COS;
wherein said second COS provides for an improved ovarian stimulation when compared to the first COS.

2. The method of claim 1, wherein optimizing the second COS comprises increasing or reducing dosage of hormone(s) administered to the human subject during COS.

3. The method of claim 2, wherein the hormone is the luteinizing hormone (LH) and/or the follicle-stimulating hormone (FSH).

4. (canceled)

5. The method of claim 1, wherein optimizing the second COS comprises aspirating follicles for assisted reproduction (AR) after a period of time shorter or longer than the period of time following hormone stimulation of the first COS.

6. (canceled)

7. The method of claim 1, wherein said at least one ovarian marker is selected from the group consisting of PDE8B, THBD, TLR2, CHODL, TGFBR2, KCNJ8, NRP1, VNN1 and combinations thereof.

8. The method of claim 1, wherein said follicular cell(s) is(are) granulosa cell(s) or cumulus cell(s).

9. The method claim 1, comprising assessing expression of at least two ovarian markers.

10. The method of claim 9, wherein said at least two ovarian markers are from at least two different sources of biological material.

11. The method of claim 1, further comprising the step of comparing the expression level of said at least one ovarian marker with a control expression level.

12. The method of claim 1, wherein the human subject is a women suffering from polycystic ovarian syndrome (PCO) or hyper stimulation syndrome (HSS).

13. A method for assessing maturity of a mammalian ovarian follicle, said method comprising assessing expression of at least one ovarian marker from said follicle, wherein said ovarian marker is selected from the group consisting of the genes listed in Tables I, II and III, and combinations thereof; and wherein said expression is indicative of follicular maturity status.

14. The method of claim 13, wherein said at least one ovarian marker is a granulosa cell marker which is expressed in granulosa cells comprised in said follicle, and wherein said granulosa cell marker is selected from the group of genes listed in Table I and combinations thereof.

15. The method claim 14, wherein said granulosa cell marker is selected from the group consisting of PDE8B, THBD, TLR2, CHODL, TGFBR2, KCNJ8, NRP1, VNN1 and combinations thereof.

16. (canceled)

17. (canceled)

18. The method of claim 13, wherein assessing expression of said at least one marker comprises measuring polynucleotide and/or polypeptide expression levels for said marker.

19. The method of claim 18, comprising measuring DNA and/or mRNA levels of a polynucleotide encoding said at least one marker.

20. The method of claim 19, wherein said polynucleotide comprises a sequence as set forth in NCBI for the GeneID numbers provided in Tables I, II and III.

21. The method of claim 20, comprising measuring expression levels of a polypeptide encoded by said at least one ovarian marker, wherein said polypeptide comprises an amino acid sequence as set forth in NCBI for the GeneID numbers provided in Tables I, II and III; or said polypeptide is encoded by a polynucleotide sequence according to the polynucleotide sequences as set forth in NCBI for the GeneID numbers provided in Tables I, II and III.

22. The method claim 13, comprising assessing expression of at least two ovarian markers.

23. The method of claim 13, further comprising the step of comparing the expression level of said at least one ovarian marker with a control expression level.

24. The method claim 13, further comprising selecting oocytes originating from follicles having a desired maturity for in vitro fertilization (IVF) and/or in vitro maturation (IVM).

25-27. (canceled)

28. A method of assessing maturity of a mammalian ovarian follicle, said method comprising:
(a) assessing in an oocyte and/or in follicular cell(s) originating from said follicle an expression level of at least one polynucleotide, wherein said at least one polynucleotide comprises a nucleotide sequence as set forth in NCBI for the GeneID numbers provided in Tables I, II and III and combinations thereof; and
(b) comparing the expression level of said at least one polynucleotide with a control expression level;
wherein a differential between expression level of said at least one polynucleotide and the control expression level is indicative of follicular maturity status.

29. The method of claim 28, comprising assessing expression level of at least one gene selected from the group consisting of PDE8B, THBD, TLR2, CHODL, TGFBR2, KCNJ8, NRP1, VNN1 and combinations thereof.

30-49. (canceled)
